SEQUENCE LISTING (5 sequences)

SEQ ID NO: 1 - DNA, 29151 bp, Artificial Sequence
  Nucleotide sequence of PP2Ce knockout mouse, wherein the PP2Ce locus was ruined
tgcatggaggcccagaaaggacattgtgtgacgttatggaggtaaagcctggttgccatgcagaccctccctaagagatgtcagggctgtgggatatctgtcgggaaaagctgctaacagggactggacccgctcaaagggaaaaagcatactgcagtcagcaaagctgaaaggcgttggagaactgaagaacattttgacatcagacatggagatgcagagtttggagtttgcccagctaggttttggtcttcctttggtccagtattttcttactatgctccctttcccacattttggaacagtgatgcatatcctatgccattatatgttgtaaatgtgtaatctttttgatttttcggggaattacagttaagagattgcattagtctttgaacttttaaacaactttggggttttatacactatggggacttttgaagttggattaaatacattttgcattacaagtctatgggggccagggagtggtggtttgaatgtgattggcccccatagggccataggaagtgtggcctttgctggaggaagtgtgtctctgtggaggtgggctttgaggtcatatatgctcaagttttgcccagtgtgacacagagtctcctctgtcacctgtagattaagatgtagagctctcagtcccttctccagcactatgtctacctgtgtgctgccatgcgtcctgtcatgatgataatggactaaacctctaaactgtaagccatccccaattaaatgatgttttctctaagagttgccatggccatggtgtcttttcacagcaatagaacccttagacagcagttctatgtctacatgaacattgtgtagcttagtctttaatcccccattatgggggctgaagttccaactgcagcagaatccatgatatctgcctccaccaataaggaaatgagaatggggttggctcattatgcacagggaataccagactctagatttaggtggtcctgccccctttccactaaagttggacatctgtggtataacatgtatgaggatgtaaaggaatgggttctccttcagtgcaagcaagtcagaagatgtacatttggttaagacattagtgcaagcaagccagatggtctttggagcctggaaataatttaatgaccattcctggaattgacctcttgtatatggctatagaaatctgcatagggggagctatttaggagtatcttggtgacattaagtagggacgctttcctggaggtccctcagggtgagcttagttcggtctcatttacagctattatggttggaagtggagcttatggagggttagggttcgtgcatagctagcagtcttgacctttatttggctaaaaatggtcaaggaactgatacgctactttttgcatcctgaacatggagggtttgtattccactgccttctcaatctttgtctttgagatagatccaaaggaatttctgggaaaatagaaattttgcagcttgtcatcttagggagctcataatccaagtgtcccaagacaggttggtttggactactgagccagacaactctaggtgcaagtttgttttgaatagtgaatttgctgccaagatctggaccatttaggtataacccaactctccaaattctaccatttttctaataacaagttttggcttcctgccacgacataacttacatggatatggggttgcaaagtacctacataggtttagtaaccccactaagcacaatggaagcatggcgggggggctgtcatctgggcaggcatagttctggatacatcaaagccccttctcttcaccagtgtacacacagccatgagggcatgctccccctcaggaatgagctaacccccccccccacacacacacacacagtaacttctgggtgtaatattttatagaaatctacttgaaaggtaggtcgggtctcagtggatgggagggatataagctccctcgttctggttttagctggatgtggtgggtctacagaatgaccttgggaatactggtgaccttcattgttattttgggtaggattctgtttacccatataaggtctccagactgggactcgtgggcaggctcagatgcaagcaagggaacagtcctctagggttattgttgggcctgtgaggacttgaggaggaaatggttccaactgctggtctctaagctgggaaagcagctaatctcattaaggtaaatccctccccttgtgggatgcagggagcccaaagcaaatcaaagggaggaggtcaatccactcttcacaggattctaataagagttttattgttttgctgtttatctttctacctgttgtggtgatttgaataggcttggcccatggcaagtgctattactaggaaatgtggtcttgttggaatatgtgtggccttgttggagcatgtatgtcactgtgggagtgggctttgaggtcctatgctcaaactccatcccatgcagacacagtctctcctggttgccttccaaccaagatgtagaactctcagttccttctccaccactatgtctgccttgacactgccatgcttccttccacaataataatggactaattacctaaaacaggctgtaagccagccctgattaaatactttcttttttaagagttaccttggtcatggtgtctctttacagcaatgaaactctaactatgacacctgtccagaattttatagtctgtaagcacattgaaaaattcagtctatgtctaaagttttagatattaattgagaggttttagctatgaaaacttggccattgtcagacccctttgctaggatgaggccaatttgggggaccagttcttggaggacctttatagatactatttgagcagtttcagaataggtgggaaacacttctacccactgggaaaaggtatttataaaaactggccagtatttatatcctcgggtggcagggctggatctcagtttctccatgttctctgggatgctggtctctcatctggactccttcctgggtaaggactctttgttttggattcacttgaacaccaatctggcactggttgagacatctggcaccaggctgtagagtgcagctgtagcataccgtgatctgagcagctcaaagttctgtggatcctgatgagtaccctgttggaggttagttaccagagtcccatcaactgtttctgggagaatgatcttttcctttagtgtcctccatcagcctgtgggttcaaactgtccatttttttcctttttgcatattttaattcctcttggttgtaccatagagtctctctgggtcaggtcaccagaatatcagtatcattggtattgttgggtggccagatcagtagttgattacctcttgcctctggggagtcatcattttgatattctcatcagtggaggatagcaacttgtaggggaagcctcatagccttcaggagggccaagatttcctctttgttttaatgttctttgctccagtggtgagaaaccctctttctctatagatcataccatgaacatgcatgctagtaaaagcatagatatggggatggactttcatttcatcatctgagagcctgggtcagactggtcagttctgctctctggccgaggtagctggccctaagtcttaggtcatttcagttaagtaatgacttacctggttttccttctagctgcattatttagtggagattcccctgctagacctgggagcaggttgtctactcttttcagagaacggtggggtttgagtttttcaaatatatggatcattagttgaaaaggagccataaactatgaaagagaactctcctttcccttgggtgcaacaggctgcttggagcaggcaggggagtacagagcaggcaggagagtataaagggagactatttctccctccttgtgtgtgttccatttagatgtcaggtcccataccaccaccacacacacaggaaccaggactgaaaactgaagtcaagcaattaaattagcaaacacactgggagcagagcagctgagagaagagcttggtgacctgccagagtcaaaaggattggggtccccaactagagggcgcaggtcttaagcctgagggtggggagtgttcacatcataaaaagccattggtcaatacaggaaacctagttggggtggctgcgatttcatgtttcagatggtctgaatggtcacctgattccaagagagggctagtggattccacagaagcccttgagcttttctgtgtgtgaaacagatatctgtccttctccataaaatgctgctttgggccatggtgcaccttgcccgccattgccatcagcactgtcaaaaactgaggagaagcgagaatagacgatcctcagctgacttagtgttcccactgctgtgaccaaaggaactcgggaggaaacagtcgactttactcactgctcccttagcagttcattgtcataagcagtgagggcaggaactctagcagggcaggaacctggaggcaagagctgatgctgcggccatggagggtgctgctaactggctttctccccatggtgtacacagcctgcttcttatacaatctaggaccaccagcccagaagcagccccacccaaaataggctgggctttcccccatcaatcactagttaagaaaatgcttacagccaaatcttttggaggcattagctctctcttttctgatgtcaagactgtgtcaagttcacaaaactagtcaacacaccaatatacatatatacgtgtgtgcgcacatgcacccgcacacacacacgccacagacaggttgaattatatacacttacaattaagtagactgtattaaaagcaaaatactcaaaacatcatttcctaaattgactattttagtagtacgtatattcttgactatatttatacgtttggcgaaaataccataagggtggttactgagcaactcccaacttaatgtgcagacatgaagcttgagataagctcactggttgaatatattagaagggctgtgtaacaaagaaaaactaagataaggttttcaggtagttagaagtaggtaatggtggctcattattccaaatattcctgagtctaactttgagtaatgagtgtcatttagaatcttagacattctagtattcacttgggccagtgaagcaggaagtctagttgcagatctttccctcttcctctttccctccaaatccaattccccagtctcatcccgagccctgcagcagaacacctgctgtggcttccccttgctctttgctaccacttccagtccatcacacagatcttcccttccaaacttttctcccctgctcattgctttatcccagcccccaaatccaataggcatctcctgggaaccctcaggctactctgaccagggaagcaggcaggccaaccgcaaattgttatttctccctccagtcctccaatgccagtcctctcagctctgtaacagacaaccttgcagtctctcctaattctgtccccattccaggggacctagcagatactggtggaacaccctgctttcttccctctgtggaccacctcagcacggcctccagccaatgatcattcccgtgtgtcagatccccaaacctcaaaacattttacctggaaacaccatcatcatcaccaccaccaccaccagcatcaccaccaccagcacaacagccatcttttcctgatgtcttcgttctctaaattccctgataaaaagataaactaacagaatgcatgtgaaaacatttttctgctccatccaagaaatactccttaatatcaaggacagacatcacctcggggtaaaaggatggaaaaagacatttcatgaaaatgaatctaagaagcaagctggtatggccatttcaaagtctgagcaaacatacttcagaccaaaactaatcagaagagataggaagggacactacatactcatcaaaggaaagaaaaatcctccaagaggacattgcagttcttaacacctgtgcaccagataatctgtgtaggggctggagagatggctcagcagttaagcacttactgtgcaagcagacagtcttaggaactggagtttggatcctcctgtcccatacaaactcaagtgggtgtgatggccttcccataattccagtgctcacaaggtagacacagagaatccctggatcaaactggttagccaggctaggtgtatctgggagctgtgtgttcaatcaaacctggtcttagtgactaaagtggagagtaactgaacactcccagcatcaacctgggacacacacacacacgcatgcatgctcgatcaccacacacacacacacacacacagagagagagagagagagagagagagagagagagagagagagagagagagagagagagaggagaaaaacatcttaagtttaaattagtcaggtgttccaatttcacctattagtgaccccaacatgacgcgtttttcaatagaaactgtctgtagaacacaggaagccttaaggcaatccagttattctcaggctatgtcgagaggcctttacaagaattgggtatacaaatggaatattgctggatacacctctccccaaaggggaatgttaatgatagtctattttattatgatacaagtgaaagatcctggcagaatgtaaaaggacacagaagccctgaaattggcaagatagacgttagtgttagcagaactagcttcactgatttagaaaaatagaggtgcacaatgctctggccactccttgaacctgtgtgtctgccaatgttctgaccaggtgtgtgcccattgctgcaccttcattagactctttccttgtacccctctcatacccatttcttgagaatagacattgtttagatctggaaatcccctactctcccccttctccttttccccctgagggcctataaaaactgagacctctttcccctcgaggtcgactcctctacccctgcgtgggatatgagtcgtccccagagctctggctttccctgaataaagcctcatgtggtttgcaacaagctcggtctatcgtgagttcttgggtgtccgctattgtcctgaggcctgaatgaggggctcctctcagagtctttcacaagtagatgttcatctcaaattaattaaatttttttgtggtgcccgatgttcctccaaatacttgttaatttaactttcatttggactaagcaagttaattcagtttttgcatattttattctttaaaagtatatggtgtgtgtgtgtgtgtgtgtgtgtgtgtgtgtgatgtgtgaaggttaaacaacaattttcaggagtcagttctctccttacaccatctgggtcctgggaatgaaactcaggctgggtggcaagtacctttacccactgagttatctcataaggtaaacaatgattttgtactgtggtttgaataaaaatcacccatacacccatagggagtggtatcattaggaggcgtggccttgtttgagtgggtggggccttgtcagaggaagtgcatcacctagggggtgggcaaaactttgagtctcgcagggcagtggaggtgtacacctttaatcccagcactagggaagcaaaggcaggcagatttctgagttcgaggctagaccggaaaaaactttgagtctcagaaactcaatccaggtctagtggttttttttttttttttttttttttttgtcactttctgctgcctctgaatccagatgtagagttctcaggtccctatctagcaacatgtctggctgtatgccaccatgcttcccaccatgacaaaaacagactaaacctctgaactgtaagccagctccagttaaatgcttttcttcataagagttgctgtggtcacgatctctctttaacctagttgagatgtatgtttctgggcttcgtgcatccaggcaagtgctctctctacttttgagccacttctccagccaaatagcaacaacaattgttttaagcccattatttgttttccatgcacgtgcttgtgtatatgtgagtacacatgagggtcccgcagataccagaaggtgtagcatagcctggagctcaagtttcaggcagttgtgagctccctaactgggctctgctgcactgagacatctctccagccccagtcaataattcccaagaggtattttcttccctttctgctccactccccaacagtttgttttgtttttccagttaacatcggcatttagcaacgttttagaagattattaaagacaaggctaaaaaaagtggatgacggtttcttaggaaaagtggtttacatgaacagatttggtgcaatgctgtgttttattccaaacacaggtacccttgatctatttctgtagttaatataaagacttgggttaccaacaacaatcttatgcttcttaaggacagtaaaataaaaattatgttgtgcccgaatattagtgttatgaaattaattcttcatttgacaaaacacgtgggatattaaaataagctcgaattgatagactataacatttacttaaaatgttgaatttcttttcttagtggtacaactaaaacattcattccactccccaccccccaacaccatttaaaactaagatttattatcaaattttgcatctggaaatgtttgagaggatgcgctgaagattaatatggcttaactcttatggccgtgtcagctgtttactgaaagccacttttaggacagaacgcgcacggcccttgcattgaggtgctttagtcacgtggttggtggggctataaatagaagtaactttttcaaaaacatttcgaactatgaattataatattgtacattttcattccctactgtctcgatatttcacccccatgggtatccctggagagacttggatacacacacacaccatgaggcaatacatatgcaaatcatgagattgttcatcaaaataaaacaacaacaacaacaacaacaacaacacacacacacacacaaacaaatgaaaccacaaaacttagcaactagatacttctgtgtggaggatgagtatactagttgtactgaagtaaaatgatcgatttgtagctatatagcaacatgtgaggctgtggaaatacaactgtaggtaaacaggcaagtgttagaggactgtgtacagccagtcaatactcacaaaggccatgaaaggggataaatgctagttctatttctttccactcttccttccttcctttctttttgtggtggatactatataaaaataggattgacagtgaaagtgtgtaagcctctaagtgaacctccacggcttcagaatggcctaactctacagaagttcatggagagaaacagagctcgggactgggcaagcatgtgttcgcgtgactacataaatctagctgtgtgatggttttatttgtaagttcattgtaatacagttattttaagaaaaagaaagcaaaccaaaataacaaaaacaaacccaggcaaaaatttgagcataaataattcagggatgtagacatattgcttggtacaatagcaagggaggatgggtagacaaggcagacaggagaaaacctcatggctggtacagtctgcttttcaaccagatcgttggcttatgctcatagtatttatgcaggttgtacttcataataatgctttaaggtaccttcatatgtatagttgaaaacttatacatagaaaggtatatatatatatactctagaatgtcagagaaacatcctaatcaacacaaatcacatacgtgcacagaaatgacagaaaaaaataagagcatgattcaaaatgataattgacaagtttaaaatctgcagagaaatttctgagtgatttgagatttccttcagaggttttcttttcttctttctctctctctctctctctctctctctctctctctctctctctctctctctctttctttctttctttcttttttccgagacagggtttctctgtgtagccctggctgtcctggaactcactctgtagaccaggctggtctcaaactcagaaatctggccgcctctgcctcccaagtgctgggattaaagggatgggttgccactgcctggcataggttttcctttaaaagcttgtttttagatgtatttacttttatgtgtgtgagtgttttggctgcatgcatatatgtgcaccatgtgtattgcctggtgcccttggaggacagaagtgggtatcaaatttcctggaagtggagttatggatgggtgtgagttactatgtgggtgctgggaggtgaacctgggtcctctgtaagagcaacaagtgttcttaaccactggtccatctctcagcttctacttcaaaggttttatcacagtcagtcagccaaaggcttagaagataaaccacgttagatgtttataacttcttacaaggtagtaaaccccaaaatagatgaggacataatgaaacattatactagagattatgtttgcttcacggttattttttgagacaaggtctcacactaaatttggaactaaccatttggctggtttgcaagtcccagggctcaccctgtctccagtgctccgataatgggatcataggtgcatgcccagcttttgacactggtgattgtgctgtggtgatttgaatgaaaatggctcccatgaactcacagagagtagcatcttgggtgtgtggccctgttggagtcggtgtgaccttgttggaggaactgtgtcactgagggtaggctttgaggtttcagaagcccaagccaggcccagtctctctctctctctctctctctctctctctctctctctctctctctctctctctctctctcctgcctgtggttactgatgcagaactctcagctacctctccagcactttgtctgccaccatgcctttatgctctctgccattttgacaatggcctaaacctctgaactctaagcaagcaagccctgttaaattattttctttctaagagttgccatggtcatggcgtctcttcacagcaaacacgacgacaggatcaaaactcatgttctcatgcttgagtgggcttgagcagcaggtacgtcaccaatagagccatctttccggtccttgccaagtaattgtcacttttctcctatctgataaaaacttaggactgccattagcttagtgttattactgagttatttgtactgtacagacgaagtagttatacaatatgcattttacaccacttctttgtttcttgtgaaatttagaaaaaaaaattcacttaagcaacagggtttttaggtggagaacaatttatttgtatttgtgtcaggtactgatagcagtttgtcccaaatctgtgtcataagaatccatgtgatttttgtggctgttgatattactgttaaaaaatgactaaatgtcattagaaaaatataaaactataccaaccatctccatgtcactgggagtcagtagatgttagagtactgcgtgatcagaattgggttcaatttcttcttggctatattgtgatattcattgtttttgggctttagccttctcctatcataaacacagataataaacatggctctgtcttcattagaagattgttgtatgggtggaaacaaatacagatatctgtaaacaactgtgcagccaggtgtggtagaacacatctgcaaaggcagaagattctatgggttcagagagagcccagtctacatgttgagttctaagacaactaaggttacacaggaagatcttgtctcccaaaaccaacaagacaaagcagttgtaaatcagaataagcaaaagtgaatttggcattagcagaaagggctttattatttttccagcacattaaacttacagtcaattataacgaaatgcttacttaggcattacacattgtaagttacatggaatttctcaacagaaatagggaaatgcctagagggtcttcaaaaaatatttttgatttttacacagatgaatcacttggatgtaaatgtcacagcataacatcacagaattgaaagatgtctaagtttacttcttaattcattgaatcaagccattatagtaatcatgcttgcttagttttggggaaacgaatcgcatacctaggtgtagcttcactaatcctagcatttggagtccaggcagaggacctttgggctgggacttttactactagtgacttcaagagaggtcagacttctggaatccttatcagtttatctacaaactgtttgttggaggagacaaaattgtatggacaaaagtactaatatacctcaaatctgctaagttattacagtaagaaataattacaattgtcctaacaatacctagacattctctctagctacagttcttagaggagttttcttttattatctgattttggaacctcttgaatgttgaatgaaatgacccattttcatattttctcgatcaggtggacaatatcgaaaactatggccataatcataattaaagacatatcctagttttcaaaaatatactcttaacagttgcttatattcctttcccatcattcctacttatgtttatattgttggggtcatattatttaaatacttaagcatgtttcttctgttgttgttgttagggaagtccaggtttttgtagaaactttagcagttacggagaatagacaaaaatctggacaaaatgtaaattgtttaaagttactttagttgccaactgtgaacattttgttatattttattgcagtaattatattttatatcttaagacggaactgggatcaaaagaaggcagaagtgtttgggggaacactggctgggagactatcaggatgcctgaccgtcagtccggtgggtcagcaccgtccttcgtggacagaggtagagagaaagagaagttccttttaaagattacttgcttcatattctaggcgaccagtccttgccccaacttcaagttatcctgctgaggtccacctttgcctactcaaactcctgccctcatcttcggcaaggactgaaggtggttgtcactttcaaggctccagaggatttctaacttgaaccataggcagagtagctcgctgagggacactccaagcatgttccttatggatgagtggccaccagcacagtcccagtcttcagaggccggcccttcctggtcctccggcgtttgccattacacgtcaagcctccggttctgagatccccggttctgccttcagagagacatagctcttgcggtccgagtccaagtcctgcctcttgggagatcccggcgggtctctgtggcagctgcctcgggtggccaaagtaatcactcactttcgatttttgagacaggagaaatgtgtcccatctcgctagccctacgaaaagtctggcgtggaccgtaccattgtgtagcgtgccggagggggggagggctcaggtacagctcccggctccccagcgagcagccgtgccgcccaagaaaccgacgttcagtgacctcacgtcactatcacattgtccctgggagccggcctccaaggcagaatcgacaatcggctagagctgggcggctggatttgccgctcaccccccgcggctctcccccgacccgcctgctgcccacgtgcgtccgccccggcgagctccttcgggcccccagccgggtgacagtgttacgcatctggaagcgccactcccggggcggggcggggcggggcgagcgtgtgcggggaggggaggggcgagcgcgagcggagccgggagcagccgagcggagtcgagccgagccgagccgagccgagctgagaggtgctggggagaggcaggcagagcctgccggcggctccgcacccatcgtgctgcgtttcccagctcggtgtccccctgcctttgcccaggcaccggcaaaacttttattgtttcggggaccccccctggatgtgtgacgaaatcgcaacgctccgctgtctcgtcccccaagctcgcctgcagcggcgatcgagggaacgtgcaaccctcgccgccgggctcttgcgccctgcgcttcctcacctgccgatcgctcccgggaaccgcgaggggggacccctaaagggaccgcgcgggcatcggcaccccgaaatcctcttcaccgttgcggaccggctcggcgcaactttaacttgattcctgtcgcccacccggggcacccgcggcggccgccgccgccgccgtcgctgcggtctcacaaagagcgcggggagggaaggcggcgcaggcggcaggcacgggccttcgggctgcccagacaaaagggaaactgccatcgttctggacgacgcggccgctctagaactagtggatcccccgggctgcaggtcgagggacctataccgttcgtatagcatacattatacgaagttatattaagggttccggatccccatcaagcttgccacaaccatggaagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaatagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctcgacggatcttgaagttcctattccgaagttcctattctctagaaagtataggaacttcagagcgcttttggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccggatcttgaagttcctattccgaagttcctattctctagaaagtataggaacttcagagcgcttttgtcgaggtcgatcgacggtatcgataagcttgatatcgaattcctgcaggtcgagggacctaataacttcgtatagcatacattatacgaagttatattaagggttccggatccccatcaagcttatcgatgatatcagatccccgggatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatcccccgggggctgcagcccgggggatccactagttctagagcggccgatttaaatacgtgctagccgagagacactggatattccgtgccattttaatataaagttcccaaggtcagaagccagttaataactgctagttttctgggaagaatgaatagtatatgcagtgaactttgtcaccaggcctcagtgtcccggttctagagtttgaggcagttaaggaacgtcgtgtctttcgtgtacccctaaaagaagaagcctgcaccagttggatagactgggtgaaggtgaaggaagctccgacttctattcgcttcgcgctagggaaggactcccagctgcaacgcttcgcttcttcccaactctgccaactttgctagagccataggacgtgggacagggtgcctcgctggtggtgggttttggctgcctggcctttgcagaggtggcagtgcgctcagagccggcgttaggcacacctgacctctatgcagtggccagcttggggatgctgatgtcctcttctttctataagcctttgcttggggttgatctgggagcaggaaatagactaaagcctgctataggtctttctggcatatttctgtgacattggacactctgggcgtgtggagtctcaggtttcctggaggtcctgcccttggccctgtgcccagacgtagggcgtgttaaaatgtggctccgtagcaagttctgacatctgcctcagctggacacttgtaacttacattgcttcaggcttccccacctctttcttggcggcattcctttcccgggttggttgtcctgtcttcctcttcccctttccttgcattcttatccgtcttcctcctcccggtgggccccagggcaccgttgcttgtttttggtctgaggaagctttcagttggttatcttataggcgctctccactctccactcaccgtttgcaaatgttgcccaggttacagtgcctccctgttaactttgtagagaacaccaaacatttggagcatttaggtgataagactggaattagtatttaaacaaacattttttcccaccccctttgcctgtttatagtatgaccttcagaatgaaagtttctcattcatcatacttttgtagttttggggcagttcggagttctcctcccattggccacagggtagctgaaagacaggaggttagttgagataggagacacagcctagtgagcaaaaagataccaacaaccaagggtagctgtttagtgatggagtcataatgttgttttatgtgcctggactgactcagtgggggtgaagcaagcttcgagtctctcccctgtaaataatttaagaaacttatatttattataagtctgcagtagaacagttgctgataatctgattctggggtttgaaagcactttagaagttttgatgacttatcagtgtggtagaaagaacacctggcggtggtgtctaatgtgacatattgtacttgtgattggatgattaaatttgtcttcagagacagaagtttcagttgaaaggtttctagcagtgtctgagcaaatacgccacccaggaaaggatgcgatggatgctttatttagttcaaagtattttaggtgcaccccactttcacgatgcacatctaaacaagtactaaaggaaaaggtgaccactggtagacaaaccgtgaaatgccttttactgcgcctagtgtttaaatacttgctgtttgcaaagtagttaacattatcagttctcacaaaaatggagtgactggcttcacctctgaccaggtgcggtcaggttagtctccttgcctttgttttaccttaggaaagaaaacccaggagtgttataaatgagctatcggggtggtgattgtaacagatactgctggtgacttctctcttgcttttattattgttattatttaaaaaactttatctgcatttattttatgctgaatttattcccaggccatgagtttttgtttcttcagtttcttctgggatatctttttctcctcctcttctggctttggaaggatctgttccctctcagtgaggatcatcactgtggcagggggagctcatgtatgggttaatccggccatgagctctgtaggtttgtcagtgcatcttaggtgccttgttcacctggatgtgttcagtgactagagaatctacatctaaacccttaagttcagcattcctctctgcatttttaagcatgtgcagcaaaaattcagcactcttttttggccaccgaccctgtgtccagccccactgtttggcctgggcgcacctaccgactccaccattatacctctggaatggcacacattgcttctttaaagtgtcgtccttcagacacttcgtggctttgcggatatgcatacacttgatggccggggcagtttcccaggtgttcttaaagtgaacacgaaggtttggacctcttgatttgcatgattttgtggggttttctgggtcaagagagtattgaaccatattcgcaggtcacctctggccgattacaggaagagcttattattattatgattttttaatatcttgttatatcattttaagacaatcccagcatctagttcctgtcctgtgtgttcttggcttcccaccagaggagggtgccagtgtggtttttttttttttttggttgatggttacaacctctcagaaaaggtgccttttctgaatataggtgcacacacttggccatacacaacagagaggttaaatgagttcaatgttatttgtgataattcatactgataaaataagccacccagatttagattcaaaaggaaactttacttgcacttgtatagttcgtccttacttattgatttcatccggctttttagacgaggtgcagttagagaaattgtgctcttcctaagctatctaaggtagtcggagactttgggtttgttggtctcttcaccagataataggcttgcatatttacctaatacgtagcactctagggatacaagggtgactgaacgtgacttctgcccgccagagaagcttcagtctaataaagagcaactgttagacccaaagcgatggcaccgggtgcttgtggtgatctaggaactgaagaacaatcatttcaagcagactgagaggtcggtgaggtgaaatgccaagaaggcaccattgactgtaagatgaagagggaggctttgcttgcctatttgtggttaaaactagggtctggctgtggagcgaaggcggtcaggagctcacaatcctcctgctttaatctgcccagtgctgtgcccatgcctctaggcatgttccacacggtgccccaaagagtgtgtgctttgacttaggttctggctttgaacctctgtgacttcatatctgctgtgaccttaggccatttgcttaagcagctttatctgggagacgtttatctaaaaaaaggtgggggtggggtggggataatgatgaatgtgaggtgaatgctcttcagtcataccttgttggaaggaatcaatggagtgaaagctaccgaagaccgctcgagcagctggaagtctgccatgttgtggcgatgataacccagggagggatggtttacctgtgggagtttcaacttgcgtcactagaaagatgtttatggggcccccgtaactaacttatttcccaaattctctgcgccttctttctaacatctggtaaacgaatgtttattagctaagggttcgatggacattgagaaagtggcactctggtctcaggctcgtggtttctgattactgagaaggactattctctgtagctaatttatacattggtaggaattctgacttttacttcagcctcctatgaattgagttagcatattcagcaagtgtccccaccccgacccccaacatatctcaggacttgtgttgaacactagactggatggccatgactttatttagatgtataaagtgtaaaatccagtagagctggtcctttccttgattctcttggaccctttaaacatcctctccaccacattatgggcagatgcttggctgcttgtgtatgtatctgaagctacgacagtgaatttcttacgtagcctaagatcaagacaatcaaaggttataaacgaagctcaggttgttgcaatagagagagaaaggttgctctttttaaacttcagtcagctaaggggctgggcttagtggcccaagtcttgtttcaagcccataggaggcaaaggcgggcggatctctgtgagttcaaaaccaatctggtctacatagtgagtttcagaacagctagaactatgtagtgagaccctgtctcgaaaaaacaacaagctaaaccaaacaaataaacaaccccctacccactatgtcatctaggaaaatgattggcacctataatttacccacaggcaaaaggcattttgtcatttgtttgtttgtgtgtttgtttgtttatttatttagtttttttgagatagggtttctctgtgtagccctggctgtcctggaactcactctgtagaccagggtggcctctaactcagaaattcgcctgcctctgcctcccaagtgctgggattaaaggcgtgcgccaccacgcccagctgtttgtttgtttgtttatttatttatttaaagtactgtacagaagaaaaatccaatcctttgcaggtttggctgacatctatccaggtgactggattttttttttttaatgctcacatgctaataggcattcctatatattataacatacatttttgaaagttcgtattccatatcagagaagaacatacgtcaggtgcttgttggggatttcagtcttgaagcccagatgcgaaactgtgtggaggcttgcctgcaggatccaggcacacaggttcctgacctgagcggtggtacttgttctgaggcaggctttcctctgagaatgttacaaaagcccagacatatatatatactccagagcaaacaggatcatgagatttggcttgttgtcaaaagtaagtggtgtatgaagcctctgttttaggctagtttaaacagaaggaaatttagagccaaaggagcgccatccccgggtccctgccgggaccgcccgccttctccagcctgtgtgcctgtctctgtgcgatgttgtcttgccggtgagggttgttctgaggcatggtcttccgaagccccgacaaacggactgcaaagacattattgagggttctcgtgcatatatagcgtacatagcatatatagtgtgcatagcatatatagcgtgcatagcatatatagcgtacatagcatatatagtgtgcatagcatatatagcgtacatagcatatatagcgtgcatagcatatatagtgtgcatagcatatatagtgtacatagcatatatagtgtgcatagcaacatagaatgcttttcatgaagaccaagggagaggtagttccctaatgcgtttccgttttcatcttaagtctgtcggatcatacaactgagtcatccggtgggtttttgtttgtttgttttgctgctataaagtgttgtgtaaatgtgtagaatgtcacaggatacattcatttggcctgttaaccccactcccaggatagctcatcttttctacattcagtttccctttgtcccacttccctctccctttgtatgcatcatttaccttgtggtataaatatgtatttatggacatttcattgggtctctctgaaataaggtccagttctaactagataaactacaagtacagtcaatggtctgcttttcaggcattacaactgagtaaatgattaactctcacatcatggtgtacaagagacctagaggtttactgaggagaaatgctttatggctttagagaaaatattattcttattttgtttaaaatggtacattaatactttcccaaaaagaaaggtctttttttcccccctttctcccttcttcgaagtctttcttgtttttcctttctcttgttcttggcaacctcttggcaagtgagtgtaggaggctggctgtgaccttgtgcatcttttaacaaactggtaagagggccaggagatggtgccaactgcgcttatctgtgtggtgtgcctcctgtggaaacagggttaagctgtgtgctgtccaaagatgagtcagtgcattctttgccccaggaagcgtgtgtgtgtgtgtgtgtgtgtgtgtgtgtgtgtgtgtgtgtgtgtgatgtgcatgtatactttcaagtgtgtgtatgtacttgcatatgtgtgtatgcatctgaaggctagaaattggtaccgcgtggactattctccactgaattactgaggtagggtctctcactgaccctggtccttgccactttggccaatatagctagccatcttgcccgtagggctgcctgtccttgcttccaaggtactcagattacagatgggacaccacacctcctaggtggtggggatctgttctgaggcaggctttcctctgagaatgttacaaaagcccagacatatatatatactccagagcaaacaggatcatgagatttggcttgttgtcaaaagtaagtggtgtatgaagcctctgttttaggctagtttaaacagaaggaaatttagagccaaaggagcgccatccccgggtccctgccgggaccgcccgccttctccagcctgtgtgcctgtctctgtgcgatgttgtcttgccggtgagggttgttctgaggcatggtcttccgaagccccgacaaacggactgcaaagacattattgagggttctcgtgcatatatagcgtacatagcatatatagtgtgcatagcatatatagcgtgcatagcatatatagcgtacatagcatatatagtgtgcatagcatatatagcgtacatagcatatatagcgtgcatagcatatatagtgtgcatagcatatatagtgtacatagcatatatagtgtgcatagcaacatagaatgcttttcatgaagaccaagggagaggtagttccctaatgcgtttccgttttcatcttaagtctgtcggatcatacaactgagtcatccggtgggtttttgtttgtttgttttgctgctataaagtgttgtgtaaatgtgtagaatgtcacaggatacattcatttggcctgttaaccccactcccaggatagctcatcttttctacattcagtttccctttgtcccacttccctctccctttgtatgcatcatttaccttgtggtataaatatgtatttatggacatttcattgggtctctctgaaataaggtccagttctaactagataaactacaagtacagtcaatggtctgcttttcaggcattacaactgagtaaatgattaactctcacatcatggtgtacaagagacctagaggtttactgaggagaaatgctttatggctttagagaaaatattattcttattttgtttaaaatggtacattaatactttcccaaaaagaaaggtctttttttcccccctttctcccttcttcgaagtctttcttgtttttcctttctcttgttcttggcaacctcttggcaagtgagtgtaggaggctggctgtgaccttgtgcatcttttaacaaactggtaagagggccaggagatggtgccaactgcgcttatctgtgtggtgtgcctcctgtggaaacagggttaagctgtgtgctgtccaaagatgagtcagtgcattctttgccccaggaagcgtgtgtgtgtgtgtgtgtgtgtgtgtgtgtgtgtgtgtgtgtgtgtgatgtgcatgtatactttcaagtgtgtgtatgtacttgcatatgtgtgtatgcatctgaaggctagaaattggtaccgcgtggactattctccactgaattactgaggtagggtctctcactgaccctggtccttgccactttggccaatatagctagccatcttgcccgtagggctgcctgtccttgcttccaaggtactcagattacagatgggacaccacacctcctaggtggtggggatcccgcctacccaccttatttatttacttatttacttatttatttctttagataggatcttgctatataggttggccccttgctccaaatcttcctgcctctttttctttttttgatttttcaagacagggtttttcttcgtagccctgtctgtcctggaacttgaattcagaaatccacatgcttctacctcgaaatcgctgagattaagggcaatcttcgctccaccctcttttgctttttaagatcagaaaagtagcttagactgtttaactgcggtgcataatttgaagcatagcaagttgctcaacacgggtggatcctgcagggatgctgctttcggaagaacctggtgggggatgttagtccctgaaaaaacaaacaaacaaacaaacaaacaaacaaaatcaactttagaggaagaaagctctatttccgctcatgtctcacaggtctcagcctggcatcactgccttcagtctgtagcagacaggacacatcacatggcacaggatgtgtcatggtggaagggcaaggcagaagacacctgcgttcctcagggcagccgataagtggagaatacggagataagatatacccttaactcatctaggtcctcagagcaacccgatcagctgtgaactcaccgatggttaattagtgccttcatggtccagtcacctcctcaccacatcaccacgtgaggactaagccaccagcacatgagccttcaggggacatttcatttgggtgaaacacacacacacacacacactttttctcccatgtgttggggattgaacccaggactttgtgtttggtaactgtgtacttcactactaagccctatctttagtcctctgtccaatttctagtgcaagtatgtggatattatcggaagtccttccaaggttttgtggttgcatttttgtttagttctttgagggtggatctgttccccatgcttactctaacaatgaatgaccttgaccttggaaactcaagttatcctcctgtctcagcttcttcaataacagaggctataggcaccaatcactacacatagctagacc

SEQ ID NO: 2 - DNA, 25 bp, Artificial Sequence
  Nucleotide sequence of knockout mouse primer, 5arm REW primer
ggaaactgccatcgttctggacgac

SEQ ID NO: 3 - DNA, 25 bp, Artificial Sequence
  Nucleotide sequence of knockout mouse primer, 3arm REW primer
cgagagacactggatattccgtgcc

SEQ ID NO: 4 - DNA, 3179 bp, Artificial Sequence
  Nucleotide sequence of promoter-less lacZ gene
aagggttccggatccccatcaagcttgccacaaccatggaagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgtcgaggaattcactcctcaggtgcaggctgcctatcagaaggtggtggctggtgtggcca

SEQ ID NO: 5 - DNA, 819 bp, Artificial Sequence
  Nucelotide sequence of a positive selection marker neomycin resistance gene
atgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccggatctt

(19) United States
(12) Patent Application Publication
(10) Pub. No.: US 2009/0094709 A1
(43) Pub. Date: Apr. 9, 2009

(54) Use of protein phosphatase 2Ce (PP2Ce) having dephosphorylating action on AMPK

(76) Inventor: Rie Kasano, Miyagi (JP)

Correspondence Address: THE NATH LAW GROUP, 112 South West Street, Alexandria, VA, 22314, US

(21) Appl. No.: 11/798,774

(22) Filed: May 16, 2007

(30) Foreign Application Priority Data
Nov. 29, 2006 (JP) P2006-321835

(51) Int. Cl. A01K 67/027; C12N 9/16; C12N 5/10; C07K 2/00

(52) U.S. Cl. 800/14; 435/196; 530/300; 435/325; 800/18

(57) ABSTRACT

A drug includes, RNA interference with protein phosphatase 2Cε (PP2Cε) as an active ingredient. According to the present invention, the activation and deactivation of AMPK can be regulated.

### CROSS-REFERENCE TO RELATED APPLICATION AND INCORPORATION BY REFERENCE

This application claims benefit of priority under 35 USC 119 based on Japanese Patent Application P2006-321835, filed Nov. 29, 2006, the entire contents of which are incorporated by reference herein.

### BACKGROUND OF THE INVENTION

1. Field of the Invention

The present invention relates to use of protein phosphatase 2Cε (PP2Cε) having a dephosphorylase action on AMPK. More specifically, the present invention relates to a drug usable for diseases such as type 2 diabetes mellitus, metabolic syndrome, cancer, arteriosclerosis, liver disease and pancreatic disease.

2. Description of the Related Art

AMP kinase (AMPK) refers to a serine/threonine kinase which is activated upon detection of a decrease in intracellular energy (or an increase in the AMP/ATP ratio) It is known that AMPK is activated by various stress stimuli such as contraction of skeletal muscles, oxygen deprivation (hypoxia) and glucose deprivation (hypoglycaemia). It was recently revealed that AMPK is also activated by leptin or adiponectin, that is, a hormone having insulin-sensitive potentiation, or by a thiazolidine derivative or metformin used as an antidiabetic agent.

Activation of AMPK promotes fatty acid β oxidation in skeletal muscles and liver to reduce the content of intracellular fat, resulting in improvement in insulin resistance generated in these organs. Further, AMPK also have various metabolic regulatory actions such as suppression of gluconeogenesis in the liver, decreased fatty acid synthesis, and promotion of glucose utilization by skeletal muscles and thus attracts lots of attention as a new molecular target agent for type 2 diabetes mellitus.

AMPK attracts an attention because it plays a central role in energy metabolism regulation and thus not only acts as a new molecular target agent for type 2 diabetes mellitus but is also related to development of metabolic syndrome and cancer. As AMPK activators, AICAR (5′-aminoimidazole-4-carbox-amide-1-β-D-ribofaranoside), metformin and a thiazolidine derivative (TZD) are known (see “Igaku No Ayumi”, Vol. 208, No. 5 (2004), pp. 313-317). Any of these activators aim at promoting the phosphorylation of AMPK thereby promoting the activation of AMPK.

In these AMPK activators, however, there is much room for improvement because an effect inherent in AMPK on weight loss is not observed although the blood-sugar level is reduced to a certain extent. It is reported that at a time when people are eating to their hearts' content, the inactivation of AMPK leads to induction of metabolic syndrome and cancer (see Trends Pharmacol Sci., Vol. 26 (2005), pp. 69-76). However, the conventional AMPK activators cannot be said to sufficiently meet demand for control of such diseases.

### SUMMARY OF THE INVENTION

A first aspect of the present invention inheres in, a drug including, RNA interference with protein phosphatase 2Cε (PP2Cε) as an active ingredient.

A second aspect of the present invention inheres in, a drug including a vector, wherein a genetic nucleic acid sequence capable of expression for PP2Cε is knocked-out.

A third aspect of the present invention inheres in, a therapeutic method for treating AMPK-mediated signal-derived diseases in nonhuman mammals, including inhibiting the association of protein phosphatase 2Cε (PP2Cε) with AMP kinase (AMPK).

A fourth aspect of the present invention inheres in, a protein including a genetic nucleic acid sequence, wherein a genetic nucleic acid sequence capable of expression for PP2Cε is knocked-out.

A fifth aspect of the present invention inheres in, a peptide including a genetic nucleic acid sequence, wherein a genetic nucleic acid sequence capable of expression for PP2Cε is knocked-out.

A sixth aspect of the present invention, a nonhuman mammal including a genetic nucleic acid sequence, wherein a genetic nucleic acid sequence capable of expression for PP2Cβ is knocked-out.

A seventh aspect of the present invention, a cell strain including a genetic nucleic acid sequence, wherein a genetic nucleic acid sequence capable of expression for PP2Cε is knocked-out.

A eighth aspect of the present invention inheres in, a vector including a genetic nucleic acid sequence, wherein a genetic nucleic acid sequence capable of expression for PP2Cε is knocked-out.

A ninth aspect of the present invention inheres in, a use of protein phosphatase 2Cε (PP2Cε) as a phosphatase that directly dephosphorylates and activates AMPK.

### BRIEF DESCRIPTION OF THE DRAWINGS

FIG. 1 shows signals around AMPK.

FIG. 2 shows construction and homologous recombination of a target (Targeting Vector/TV).

FIG. 3 shows a profile of a homologously recombined ES clone by Southern blotting.

FIG. 4 shows a profile of genomic DNA purified from a mouse tail by Southern blotting.

FIG. 5 shows wild-type (PP2Cε+/+) (in the left) and homo (PP2Cε−/−) (in the right) litter male mice respectively within 24 hours after birth.

FIG. 6 shows 1-week-old wild-type (PP2Cε+/+) (in the left) and homo (PP2Cε−/−) (in the right) litter male mice respectively.

FIG. 7 shows 4-week-old wild-type (PP2Cε+/+) (in the left) and homo (PP2Cε−/−) (in the right) litter male mice respectively.

FIG. 8 shows 1-year-old wild-type (PP2Cε+/+) (in the left) and homo (PP2Cε−/−) (in the right) litter male mice respectively.

FIG. 9 shows the survival rate of homo (PP2Cε−/−) mice (n=100).

FIG. 10 shows a change in body weight in wild-type (PP2C+/+) and homo (PP2C−/−) litter male mice after birth until 1 year-old.

FIG. 11 shows the blood sugar level determined from 5-week-old wild-type (PP2Cε+/+) and homo (PP2Cε−/−) litter male mice given a high-fat high-caloric food for 4 weeks during eating or during 24-hour fasting.

FIG. 12 shows the insulin level determined from 5-week-old wild-type (PP2Cε+/+) and homo (PP2ε−/−) litter male mice given a high-fat high-caloric food for 4 weeks during eating or during 24-hour fasting.

FIG. 13 shows the influence of PP2Cε on activation of AMPK (in vitro assay).

FIG. 14 shows the interaction between endogenous PP2Cε and AMPK in the mouse liver.

FIG. 15 shows a change in expression of PP2Cε mRNA in the mouse liver.

FIG. 16 shows a graph of expression level of PP2Cε mRNA by mass spectroscopic analysis.

FIG. 17 shows the results of phosphorylation level of AMPKα Thr172 (upper stage), expression level of AMPKα protein (middle stage) and expression level of actin protein (lower stage) by Western blot analysis.

FIG. 18 shows the results of phosphorylation level of acetyl CoA carboxylase (ACC) Ser79 (upper stage), expression level of ACC protein (middle stage) and expression level of actin protein (lower stage) by Western blot analysis.

FIG. 19 shows the results of phosphorylation level of mammalian target of rapamycin (mTOR) Ser2448 (upper stage), expression level of mTOR protein (middle stage) and expression level of actin protein (lower stage) by Western blot analysis.

### DETAILED DESCRIPTION OF THE INVENTION

Hereinafter, the present invention is described by reference to the embodiments, but is not limited to the following embodiments. Elements having the same function or a similar function in the drawings are collectively described by giving the same or similar symbol.

### Relationship Between AMPK and Protein Phosphatase 2Cε (PP2Cε)

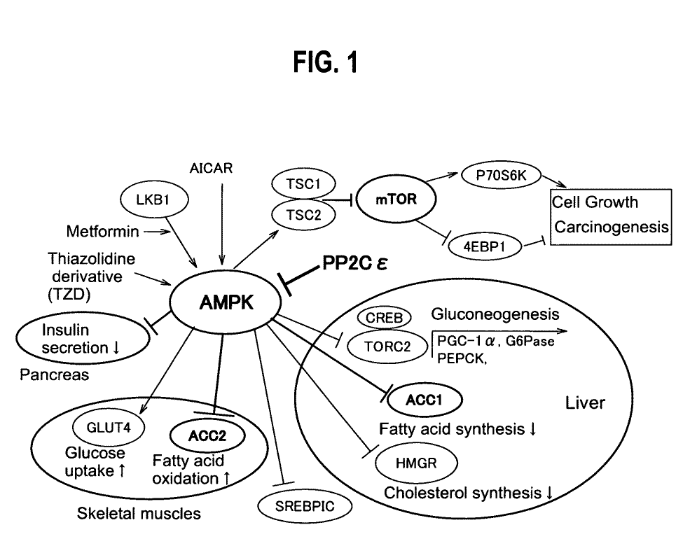

FIG. 1 shows signals around AMPK. Abbreviations in FIG. 1 are as follows:

PP2Cε: protein phosphatase 2Cε
AMPK: adenosine mono phosphate (AMP)-activated protein kinase
CREB: cAMP response element-binding protein
TORC2: transducer of regulated CREB activity 2
PGC-1α: peroxisome proliferative activated receptor-γ co-activator 1α
G6Pase: glucose-6-phosphatase
PEPCK: phosphoenolpyruvate carboxykinase
ACC1: acetyle-CoA carboxylases 1

HMGR: 3-hydroxy-3-methylglutaryl-CoA reductase

SREBP-1: sterol regulatory element-binding protein 1
ACC2: acetyle-CoA carboxylases 2
GLUT4: (insulin-responsive) glucose transporter 4
LKB1: Peutz-Jeghers syndrome gene
AICAR: 5-aminoimidazole-4-carboxamide-1-β-D-ribofuranoside
TSC1: The tuberous sclerosis complex 1
TSC2: The tuberous sclerosis complex 2
mTOR: The mammalian target of rapamycin
p70S6K: p70 ribosomal S6 kinase
4E-BP1: eukaryotic initiation factor 4E-binding protein 1

As shown in FIG. 1, ACC1 contributing to fatty acid synthesis in the liver, ACC2 contributing to fatty acid oxidation in skeletal muscles, GLUT4 contributing to sugar incorporation in skeletal muscles, and mTOR contributing to cell growth are present downstream of AMPK. Thus, AMPK plays a central role in energy metabolism regulation thereby participating in development of metabolic syndrome and cancer, thus attracting an attention (see Trends Pharmacol. Sci., Vol. 26 (2005), pp. 69-76). Since it was reported that a PP2C family member (whose subtype is not known) inactivates AMP-activated protein kinase α (AMPKα) by dephosphorylating Thr172 thereof, some study groups have made reports suggesting that the PP2C family is a negative regulator of AMPK.

For example, a report suggesting that a PP2C family member (whose subtype is not known) inactivates AMP-activated protein kinase α (AMPKα) by dephosphorylating Thr172 thereof was made in 1991 (see Eur. J. Biochem., Vol. 199 (1991), pp. 691-697). It was also reported in 1995 and 1996 that according to in vitro kinase assay in Escherichia coli, AMPK is inactivated by dephosphorylation with human PP2Cα (see FEBS Letters, Vol. 377 (1995), pp. 421-425; Biochem. J., Vol. 320 (1996), pp. 801-806). It was reported in 2004 that human PP2Cα inhibits the activation of AMPK in the rat heart (see Eur. J. Biochem., Vol. 271 (2004), pp. 2215-2224). It was revealed in 2005 that the expression of PP2C is increased in myocardial cells of a fat rat, to suppress phosphorylation of AMPK (see AJP-Endo, Vol. 288 (2005), pp. 216-221). In this fat rat, an inverse correlation of AMPK with PP2C was reported; that is, it was reported that by administering triglitazone (thiazolidine derivative) to the fat rat, the expression of PP2C in myocardial cells is decreased while the phosphorylation of AMPK is promoted (see AJP-Endo, Vol. 288 (2005), pp. 216-221).

A mouse in which ACC2 is knocked-out, even when given a high-fat calorie-rich food, hardly shows an increase in body weight and blood sugar level (see Science, Vol. 291 (2001), pp. 2613-2616; PNAS, Vol. 100 (2003), pp. 10207-10212) and in a mouse in which ACC1 is knocked-out, embryonic death is reported (see PNAS, Vol. 102 (2005), pp. 12011-12016). Also, mTOR is related to cell growth and canceration (see Trends Pharmacol. Sci., Vol. 26 (2005), pp. 69-76; Genes & Dev., Vol. 16 (2002), pp. 1472-1487). Eur. J. Biochem., Vol. 271 (2004), pp. 2215-2224 shows the in vitro inactivation of AMPK with rat heart-derived PP2Cα, which is not observed to have a physiological change or influence as compared with analysis of a mouse in which PP2Cε is knocked-out, as described in the Examples below. In AJP-Endo., Vol. 288 (2005), pp. 216-221, down-regulation of PP2C in myocardial cells and acceleration of phosphorylation of AMPK are observed after troglitazone (TGZ) belonging to the thiazolidine derivative is administered to a fat mouse. In FEBS Letters, Vol. 377 (1995), pp. 421-425, it is described that bacterially expressed human protein phosphatase-2Cα causes the dephosphorylation of AMPK. However, the relationship between down-regulation of PP2C and acceleration of AMPK activation is not proven.

Which member of the PP2C family takes a major role as an intracellular physiological AMPK inhibitory factor has been unrevealed. Accordingly, a report physiologically proves the relationship between down-regulation of PP2C and acceleration of AMPK acceleration has been desired.

The present inventors made extensive study, and as a result they found that in analysis of PP2Cε-deficient knockout mice, PP2Cε functions as a negative regulator of AMPK in cells, as will be described in the Examples below. That is, the present inventors found that observed phenotypes such as lower body weight and low blood sugar level observed in mice in which PP2Cε was knocked-out are attributable to the fact that PP2Cε acts as a phosphatase for AMPK. In the Examples, the mice in which PP2Cε was knocked-out were characterized by accelerating activation of AMPK, showing lower blood sugar level and lower insulin level, and being free from obesity even with a high-fat high-calorie food given. From this result, the physiological activity of PP2Cε was shown. Even at the molecular cellular biological level, the dephosphorylation of AMPK with PP2Cε occurs concentration-dependently and the binding of PP2Cε to AMPK was also indicated. PP2Cε was shown to be a phosphatase that directly dephosphorylated AMPK. The expression of PP2Cε or the inhibition of binding of PP2Cε to AMPK is considered to yield extremely higher in vivo physiological activity on AMPK than by metformin, AICAR or a thiazolidine derivative.

The inventors' finding revealed that when an animal is made deficient in PP2Cε, the phosphorylation of AMPK is accelerated thereby activating AMPK, which is followed by inactivation of ACC1, ACC2 and mTOR and activation of GLUT4 downstream therefrom, as shown in FIG. 1. Such interaction of PP2Cε with AMPK can be used to provide, for example, a drug, a therapeutic method and a bio kit participating in signals around AMPK. Hereinafter, the present invention is described in more detail by reference to the embodiment.

### [Drug and Therapeutic Method]

According to this embodiment, there can be provided the following drug and therapeutic method.

A drug includes an ingredient inhibiting the association of protein phosphatase 2Cε (PP2Cε) with AMP kinase (AMPK). A drug includes, as an active ingredient, RNA interference with protein phosphatase 2Cε (PP2Cε). The RNA interference with PP2Cε includes RNAi, siRNA, and shRNA. A drug includes a vector wherein a genetic nucleic acid sequence capable of expression for PP2Cε is knocked-out. An SNALP capsule includes RNA interference with PP2Cε. A vector wherein RNA interference with PP2Cε is integrated. A vector wherein RNA interference with PP2Cε is integrated in Sendai virus.

A therapeutic method for AMPK-mediated signal-derived diseases, which comprises inhibiting the association of protein phosphatase 2Cε (PP2Cε) with AMP kinase (AMPK). A therapeutic method for AMPK-mediated signal-derived diseases, which comprises use, as an active ingredient, of RNA interference with protein phosphatase 2Cε (PP2Cε). A therapeutic method for AMPK-mediated signal-derived diseases, wherein the RNA interference with PP2Cε is selected from the group consisting of RNAi, siRNA, and shRNA. A therapeutic method for AMPK-mediated signal-derived diseases, which comprises applying to an affected area a vector wherein a genetic nucleic acid sequence capable of expression for PP2Cε is knocked-out.

The drug, SNALP capsule, vector and therapeutic method described above can suppress the action of PP2Cε as AMPK dephosphorylase and can thus regulate the dephosphorylation of AMPK. Accordingly, the drug, SNALP capsule, vector and therapeutic method described above can be used for AMPK-mediated signal-derived diseases.

Specifically, the drug in this embodiment can accelerate the activation of AMPK more significantly than by conventional drugs such as AICAR (5-aminoimidazole-4-carboxamide-1-β-D-ribofuranoside), metformin and a thiazolidine derivative (TZD) and is thus usable as a therapeutic agent for type 2 diabetes. The drug in this embodiment can also be used as a prophylactic agent for metabolic syndrome and cancer and as a therapeutic/prophylactic agent for arteriosclerosis. The drug in this embodiment can also be used for liver disease, pancreatic disease etc. The SNALP capsule, the vector and the therapeutic method, similar to the drug, can also be used for various diseases.

As used herein, the “metabolic syndrome (metabolic disorder syndrome)” refers to combined lifestyle-related diseases also called syndrome X (Reaven, 1988), deadly quartet (Kaplan, 1989), the insulin resistant syndrome (De Fronzo, 1991), or the visceral fat syndrome. The “metabolic syndrome”, each elements of which is not a disease, constitutes a “definite” disease upon combination of its elements. The metabolic syndrome, while overlapping with “obesity”, “hypertension”, “hyperglycemia” and/or “hyperlipemia”, can be developed in some cases. Such patients are liable to myocardial infarction or cerebral infarction. The obesity referred to above is upper-body obesity, specifically visceral fat accumulation. Hyperinsulinemia is also observed.

Major diagnostic criteria for the metabolic syndrome include US Hyperlipemia Treatment Guideline and Diagnostic Criteria by World Health Organization (WHO). The US Hyperlipemia Treatment Guideline (ATPIII: Adult Treatment Panel III, NCEP National Cholesterol Education Program) stipulates that a person is diagnosed as having the metabolic syndrome when the person meets 3 of 5 criteria below: (1) waist is 102 cm or more for men (in the case of Japanese, 85 cm or more) or 88 cm or more for women (in the case of Japanese, 90 cm or more), (2) neutral fat is not less than 150 mg/dl, (3) HDL cholesterol is less than 40 mg/dl for men or less than 50 mg/dl for women, (4) systolic blood pressure is 130 mmHg or more, or diastolic blood pressure is 85 mmHg or more, and (5) fasting blood sugar level is not less than 110 mg/dl.

The Diagnostic Criteria by WHO stipulate that a person is diagnosed as having the metabolic syndrome when the person not only has hyperinsulinemia (the top 25% of nondiabetic patients) or a fasting blood sugar level of not less than 110 mg/dl but also meets 2 criteria out of the following criteria: (1) visceral obesity waste/hip ratio >0.9 (male) or >0.85 (female), or BMI is 30 or more or waist is 94 cm or more, (2) abnormal lipid metabolism: neutral fat is not less than 150 mg/dl or HDL cholesterol level is less than 35 mg/dl (male) or less than 39 mg/dl (female), (3) high blood pressure is not less than 140/90 mmHg, or an antihypertensive is used, and (4) microalbuminuria (urinary albumin excretion rate is not less than 20 μg/min, or the urinary albumin/creatine ratio is not less than 30 mg/g·Cr).

In addition to the criteria described above, Metabolic Syndrome Diagnostic Criteria in Japan, set up by “Exploratory Committee for Metabolic Syndrome Diagnostic Criteria” composed of members of Japan Atherosclerosis Society, Japan Diabetes Society, Japanese Society of Hypertension, Japanese Circulation Society, the Japanese Society of Nephrology, the Japanese Society on Thrombosis and Hemostasis, Japan Society for the Study of Obesity, and Japanese Society of Internal Medicine, stipulate that a person is diagnosed as having the metabolic syndrome when the person not only meets the following requirement (1) but also falls under 2 or more requirements out of the following requirements (2), (3) and (4): (1) waist measurement: 85 cm or more for men or 90 cm or more for women, (2) blood lipid (abnormal lipid metabolism): neutral fat (triglyceride) level is not less than 150 mg/dl and/or HDL cholesterol (high-density lipoprotein cholesterol) level is less than 40 mg/dL, (3) blood pressure: systolic blood pressure is 130 mmHg or more, and diastolic blood pressure is 85 mmHg or more, and (4) blood sugar (sugar metabolism) fasting blood sugar level is not less than 110 mg/dl.

Because the metabolic syndrome refers to combined lifestyle-related diseases, the diagnostic criteria cannot be always unambiguous, so the metabolic syndrome should be judged according to diagnostic criteria which on the basis of human race, residence etc., are selected from the diagnostic criteria described above.

### [Therapeutic Agent for Liver Disease and Therapeutic Method for Liver Disease]

Liver disease can be treated by introducing RNA interference (RNAi, siRNA, shRNA etc.) with PP2Cε into the liver. For example, RNA interference (RNAi, siRNA, shRNA etc.) with PP2Cε is encapsulated in stable nucleic acid lipid particle (SNALP) capsules and then intravenously injected. This technique is reported in a study where siRNA is encapsulated in stable nucleic acid lipid particle (SNALP) capsules in order to cause silencing of previously stable apolipoprotein B (ApoB) and then administered intravenously to a cynomolgus monkey in a dose of 1 or 2.5 mg/kg (see Nature, Vol. 441 (2006), pp. 111-114).

### [Therapeutic Agent for Skeletal Muscles and Therapeutic Method for Skeletal Muscles]

Skeletal muscles can be treated by introducing RNA interference (RNAi, siRNA, shRNA etc.) with PP2Cε into a skeletal muscle or adipose tissue. For example, RNA interference with PP2Cε is integrated in a virus vector and then injected directly into a skeletal muscle or adipose tissue. This technique wherein an HGF (hepatocyte growth factor) gene previously integrated in Sendai virus is injected into femoral muscle was developed by Ryuichi Morishita and clinically applied as therapy for arteriosclerosis obliterans, (see “Myakukangaku (Angiology)”, Vol. 44 (2004), No. 3, pp. 85-98; “Myakukangaku (Angiology)”, Vol. 44 (2004), No. 4, pp. 145-150).

Where the drug in this embodiment is used as the prophylactic/therapeutic agent, the drug is advantageously used on a purified level of at least 90%, preferably at least 95%, more preferably at least 98% and most preferably at least 99%.

The drug in this embodiment can be used orally, for example, in the form of tablets which may be sugar coated if necessary and desired, capsules, elixirs, microcapsules etc., or parenterally in the form of injectable preparations such as a sterile solution and a suspension in water or with other pharmaceutically acceptable liquid. These preparations can be manufactured by mixing the drug in this embodiment with a physiologically acceptable known carrier, a flavoring agent, an excipient, a vehicle, an antiseptic agent, a stabilizer, a binder, etc. in a unit dosage form required in a generally accepted manner that is applied to making medicines. The active ingredient in the preparation is controlled in such a dose that an appropriate dose is obtained within the specified range given.

Additives miscible with tablets, capsules, etc. include a binder such as gelatin, corn starch, tragacanth and gum arabic, an excipient such as crystalline cellulose, a swelling agent such as corn starch, gelatin, alginic acid, etc., a lubricant such as magnesium stearate, a sweetening agent such as sucrose, lactose or saccharin, and a flavoring agent such as peppermint, akamono oil or cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredient in a vehicle such as water for injection, with a naturally occurring vegetable oil such as sesame oil, coconut oil, etc. to prepare the pharmaceutical composition.

Examples of an aqueous medium for injection include physiological saline and an isotonic solution containing glucose and other auxiliary agents (e.g., D-sorbitol, D-mannitol, sodium chloride, etc.) and may be used in combination with an appropriate solubilizer such as an alcohol (e.g., ethanol or the like), a polyalcohol (e.g., propylene glycol and polyethylene glycol), a nonionic surfactant (e.g., polysorbate 80™ and HCO-50), etc. Examples of the oily medium include sesame oil, soybean oil, etc., which may also be used in combination with a solubilizer such as benzyl benzoate, benzyl alcohol, etc. The drug in this embodiment may further be formulated with a buffer (e.g., phosphate buffer, sodium acetate buffer, etc.), a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, etc. The thus prepared liquid for injection is normally filled in an appropriate ampoule.

The vector in which the genetic nucleic acid sequence capable of expression for PP2Cε in this embodiment is knocked-out may also be prepared into medicines in a manner similar to the procedures above, and such preparations are generally used parenterally.

Since the thus obtained medicine is safe and low toxic, and can be administered to, for example, warm-blooded animals (e.g., human, rat, mouse, guinea pig, rabbit, chicken, sheep, swine, bovine, horse, cat, dog, monkey, chimpanzee etc.).

The dose of the drug in this embodiment may vary depending on target disease, subject to be administered, route for administration, etc. When the drug in this embodiment is orally administered for example for the purpose of treatment of arteriosclerotic disease, the drug is administered to adult (as 60 kg body weight) generally in a daily dose of approximately 0.1 mg to 100 mg, preferably approximately 1.0 mg to 50 mg, more preferably approximately 1.0 to 20 mg. When the drug is parenterally administered, a single dose of the drug in this embodiment may vary depending on subject to be administered, target disease, etc. When the drug in this embodiment is administered to adult (as 60 kg body weight), it is convenient to administer the drug by injection to the affected area, generally in a daily dose of approximately 0.01 to 30 mg, preferably approximately 0.1 to 20 mg, more preferably approximately 0.1 to 10 mg. For other animal species, the corresponding dose as converted per 60 kg weight can be administered.

The drug in this embodiment can be formed into a pharmaceutical preparation and used as a therapeutic/prophylactic agent. For example, the composition for oral administration includes solid or liquid preparations, specifically tablets (including dragees and film-coated tablets), pills, granules, powdery preparations, capsules (including soft capsules), syrup, emulsions, suspensions, etc. Such a composition is manufactured by publicly known methods and contains a carrier, a diluent or excipient conventionally used in the field of pharmaceutical preparations. Examples of the carrier or excipient for tablets are lactose, starch, sucrose, magnesium stearate, etc.

Examples of the composition for parenteral administration are injectable preparations, suppositories, etc. The injectable preparations may include dosage forms such as intravenous, subcutaneous, intracutaneous and intramuscular injections, drip infusions, intraarticular injection, etc. These injectable preparations may be prepared by methods known per se. For example, the injectable preparations may be prepared by dissolving, suspending or emulsifying the drug described above in a sterile aqueous medium or an oily medium conventionally used for injections. As the aqueous medium for injections, there are for example physiological saline, an isotonic solution containing glucose and other auxiliary agents, etc., which may be used in combination with an appropriate solubilizer such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed e.g., sesame oil, soybean oil, etc., which may be used in combination with a solubilizer such as benzyl benzoate, benzyl alcohol, etc. The injection thus prepared is usually filled in an appropriate ampoule. The suppository used for rectal administration may be prepared by blending the aforesaid drug with conventional bases for suppositories.

Advantageously, the pharmaceutical compositions for oral or parenteral use described above are prepared into pharmaceutical preparations with a unit dose suited to fit a dose of the active ingredient. Such unit dose preparations include, for example, tablets, pills, capsules, injections (ampoules), suppositories, etc. The amount of the aforesaid compound contained is generally 5 to 500 mg per dosage unit form; it is preferred that the aforesaid compound is contained in about 5 to about 100 mg especially in the form of injection, and in 10 to 250 mg for the other forms.

Each composition described above may further contain other active components unless formulation causes any adverse interaction with the compound described above.

### [Detection Method]

According to the embodiment of the invention, there is provided a method of detecting an AMPK-mediated signal-derived disease cell, which comprises detecting a change in PP2Cε gene activity in a sample isolated from a patient.

As the sample, it is possible either a biopsy tissue or a biological fluid. Examples of the sample include urine, blood, cerebrospinal fluid or saliva. The change includes an increase in PP2Cε gene activity as compared with the normal control. Preferably, the detection step comprises assaying a sample for mRNA complementary to PP2Cε DNA including polymorphism thereof, by using an assay selected from the group consisting of in situ hybridization, Northern blotting, and reverse transcriptase-polymerase chain reaction.

The detection step also preferably assays a sample for PP2Cε gene product including polymorphism thereof and a peptide fragment thereof, by using an assay selected from the group consisting of immunohistochemical and immunocytochemical staining, ELISA, RIA, immunoblotting, immunoprecipition reaction, Western blotting, functional assay, and protein-shortening test.

Preferably, the detection of PP2Cε gene activity in a sample determines a change in the phosphorylation pattern of a protein influenced by PP2Cε gene product. The AMPK-mediated signal-derived disease includes, but is not limited to, type 2 diabetes mellitus, metabolic syndrome, cancer, arteriosclerosis, liver disease and pancreatic disease.

### [Bio Kit]

According to the embodiment of the invention, there are provided the following bio kits:

A kit for detecting PP2Cε activity, includes a molecular probe complementary to a genetic sequence of PP2Cε mRNA, a means for detecting hybridization of the molecular probe with mRNA, and a detection means showing the activity of PP2Cε gene. A kit for detecting a gene product accompanying PP2Cε gene activity, includes a drug mimicking a natural protein binding to PP2Cε gene product and a detection means showing the presence of the gene product by detecting the binding of the drug thereto.

### [Knockout Protein Etc.]

According to the embodiment of the invention, there are provided the following knockout protein etc.:

A protein or peptide wherein a genetic nucleic acid sequence capable of expression for PP2Cε is knocked-out. A nonhuman mammal or cell strain wherein a genetic nucleic acid sequence capable of expression for PP2Cε is knocked-out. A vector wherein a genetic nucleic acid sequence capable. of expression for PP2Cε is knocked-out. A mouse wherein a genetic nucleic acid sequence capable of expression for PP2Cε is knocked-out.

In this embodiment, the nonhuman mammal or cell strain wherein a genetic nucleic acid sequence capable of expression for PP2Cε is knocked-out refers to a nonhuman mammalian embryonic stem cell that suppresses the ability of the nonhuman mammal to express the DNA by artificially mutating the DNA in this embodiment possessed in the nonhuman mammal, or the DNA has no substantial ability to express the protein in this embodiment (hereinafter sometimes referred to as the knockout DNA in this embodiment) by substantially inactivating the activities of the protein in this embodiment encoded by the DNA (hereinafter merely referred to as ES cell).

Examples of the nonhuman mammal that can be used include bovine, swine, sheep, goat, rabbits, dogs, cats, guinea pigs, hamsters, mice, rats and the like. Above all, preferred are rodents, especially mice (e.g., C57BL/6 strain, DBA2 strain, etc. for a pure line and for a cross line, B6C3F₁ strain, BDF₁ strain, B6D2F₁ strain, BALB/c strain, ICR strain, etc.) or rats (Wistar, SD, etc.) and the like, since they are relatively short in ontogeny and life cycle from a standpoint of creating model disease animals, and are easy in breeding.

“Mammals” in a recombinant vector that can be expressed in mammals include human etc. in addition to the aforesaid nonhuman mammals.

Techniques for artificially mutating the DNA in this embodiment include deletion of apart or all of the DNA sequence and insertion of, or substitution with, other DNA, e.g., by genetic engineering. By these variations, the knockout DNA in this embodiment may be prepared, for example, by shifting the reading frame of a codon or by disrupting the function of a promoter or exon.

Specifically, the nonhuman mammalian embryonic stem cell, in which the genetic nucleic acid sequence capable of expression for PP2Cε in this embodiment is inactivated (hereinafter merely referred to as the ES cell with the DNA in this embodiment inactivated or the knockout ES cell in this embodiment) can be obtained by, for example, isolating the DNA in this embodiment possessed by the target nonhuman mammal, inserting a DNA strand (hereinafter simply referred to as targeting vector) having a DNA sequence constructed so as to eventually destroy the gene by inserting into its exon site a chemical resistant gene such as a neomycin resistant gene or a hygromycin resistant gene, or a reporter gene such as lactZ (β-galactosidase gene) or cat (chloramphenicol acetyltransferase gene), etc. thereby destroying the functions of exon, or by inserting into the intron site between exons a DNA sequence which terminates gene transcription (e.g., polyA-added signal, etc.) thereby disabling the synthesis of complete messenger RNA, into a chromosome of the animal cells by, e.g., homologous recombination. The thus obtained ES cells are analyzed by the Southern hybridization using as a probe a DNA sequence on or near the DNA in this embodiment, or by PCR using as primers a DNA sequence on the targeting vector and another DNA sequence near the DNA in this embodiment which is not included in the targeting vector, and the knockout ES cell in this embodiment is selected.

The parent ES cells to inactivate the DNA in this embodiment by homologous recombination, etc. may be of a strain already established as described above, or may be originally established in accordance with a modification of the known method by Evans and Kaufma. For example, in the case of mouse ES cells, currently it is common practice to use ES cells of the 129 strain. However, since their immunological background is obscure, the C57BL/6 mouse or the BDF₁ mouse (F₁ hybrid between C57BL/6 and DBA/2), wherein the low ovum collection per C57BL/6 mouse or C57BL/6 has been improved by crossing with DBA/2, may be preferably used, instead of obtaining a pure line of ES cells with the clear immunological genetic background. The BDF₁ mouse is advantageous in that when a pathologic model mouse is generated using ES cells obtained therefrom, the genetic background can be changed to that of the C57BL/6 mouse by back-crossing with the C57BL/6 mouse, since its background is of the C57BL/6 mouse, as well as being advantageous in that ovum availability per animal is high and ova are robust.

In establishing ES cells, blastocytes of 3.5 days after fertilization are commonly used. A large number of early stage embryos may be acquired more efficiently, by collecting the embryos of the 8-cell stage and using the same after culturing until the blastocyte stage.

Although the ES cells used may be of either sex, male ES cells are generally more convenient for generation of a germ cell line chimera and are therefore preferred. It is desirable to identify sexes as soon as possible also in order to save painstaking culture time.

As an example of the method for sex identification of the ES cell, mention may be made of a method in which a gene in the sex-determining region on the Y-chromosome is amplified by PCR and detected. When this method is used, ES cells (about 50 cells) corresponding to almost 1 colony are sufficient, whereas karyotype analysis hitherto required about 10⁶ cells; therefore, the first selection of ES cells at the early stage of culture can be based on sex identification, and male cells can be selected early, which saves a significant amount of time at the early stage of culture.

Second selection can be achieved by, for example, number of chromosome confirmation by the G-banding method. It is usually desirable that the chromosome number of the obtained ES cells be 100% of the normal number. However, when it is difficult to obtain the cells having the normal number of chromosomes due to physical operation etc. in cell establishment, it is desirable that the ES cell be again cloned to a normal cell (e.g., in mouse cells having the number of chromosomes being 2n=40) after the gene of the ES cells is rendered knockout.

Although the embryonic stem cell line thus obtained shows a very high growth potential, it must be subcultured with great care, since it tends to lose its ontogenic capability. For example, the embryonic stem cell line is cultured at about 37° C. in a carbon dioxide incubator (preferably about 5% carbon dioxide and about 95% air, or about 5% oxygen, about 5% carbon dioxide and about 90% air) in the presence of LIF (1-10000 U/ml) on appropriate feeder cells such as STO fibroblasts, treated with a trypsin/EDTA solution (normally about 0.001 to about 0.5% trypsin/about 0.1 to 5 mM EDTA, preferably about 0.1% trypsin/about 1 mM EDTA) at the time of passage to obtain separate single cells, which are then seeded on freshly prepared feeder cells. This passage is normally conducted every 1 to 3 days; it is desirable that cells be observed at passage and cells found to be morphologically abnormal in culture, if any, be abandoned.

By allowing ES cells to reach a high density in mono-layers or to form cell aggregates in suspension under appropriate conditions, it is possible to differentiate them to various cell types, for example, parietal and visceral muscles, cardiac muscle or the like [M. J. Evans and M. H. Kaufman, Nature, Vol. 292, p. 154, 1981; G. R. Martin, Proc. Natl. Acad. Sci. U.S.A., Vol. 78, p. 7634, 1981; T. C. Doetschman et al., Journal of Embryology and Experimental Morphology, Vol. 87, p. 27, 1985].

The nonhuman mammal in which the genetic nucleic acid sequence capable of expression for PP2Cε in this embodiment is knocked-out can be identified from a normal animal by measuring the amount of mRNA in the subject animal by a publicly known method, and indirectly comparing the levels of expression. As the nonhuman mammal, the same examples supra apply.

With respect to the nonhuman mammal deficient in expression of the genetic nucleic acid sequence capable of expression for PP2Cε in this embodiment, expression of PP2Cε can be made knockout by transfecting a targeting vector, prepared as described above, to mouse embryonic stem cells or mouse oocytes thereof, and conducting homologous recombination in which a targeting vector DNA sequence, wherein the DNA in this embodiment is inactivated by the transfection, is replaced with the genetic nucleic acid sequence capable of expression for PP2Cε on a chromosome of a mouse embryonic stem cell or mouse oocyte.

The cells with the genetic nucleic acid sequence capable of expression for PP2Cε in this embodiment in which the DNA in this embodiment is rendered knockout can be identified by the Southern hybridization analysis using as a probe a DNA sequence on or near the genetic nucleic acid sequence capable of expression for PP2Cε in this embodiment, or by PCR analysis using as primers a DNA sequence on the targeting vector and another DNA sequence which is not included in the DNA in this embodiment derived from mouse, which is used as the targeting vector. When nonhuman mammalian embryonic stem cells are used, the cell line wherein the genetic nucleic acid sequence capable of expression for PP2Cε in this embodiment is inactivated is cloned by homologous recombination; the resulting cloned cell line is injected to, e.g., a nonhuman mammalian embryo or blastocyte, at an appropriate stage such as the 8-cell stage. The resulting chimeric embryos are transplanted to the uterus of the pseudo-pregnant nonhuman mammal. The resulting animal is a chimeric animal composed of both cells having the normal locus of the DNA in this embodiment and those having an artificially mutated locus of the DNA in this embodiment.

When some germ cells of the chimeric animal have a mutated locus of the DNA in this embodiment, an individual, in which all tissues are composed of cells having an artificially mutated locus of the DNA in this embodiment, can be selected from a series of offspring obtained by crossing between such a chimeric animal and a normal animal, e.g., by coat color identification, etc. The individuals thus obtained are normally deficient in heterozygous expression of the protein in this embodiment. The individuals deficient in homozygous expression of the protein in this embodiment can be obtained from offspring of the intercross between the heterozygotes.

When an oocyte is used, a DNA solution may be injected, e.g., to the prenucleus by microinjection thereby obtaining a transgenic nonhuman mammal having a targeting vector introduced into its chromosome. From such transgenic nonhuman mammals, those having a mutation at the locus of the DNA in this embodiment can be obtained by selection based on homologous recombination.

As described above, individuals wherein the genetic nucleic acid sequence capable of expression for PP2Cε in this embodiment is rendered knockout permit passage rearing under ordinary rearing conditions, after it is confirmed that in the animal individuals obtained by their crossing, the DNA has been knockout.

Furthermore, the genital system may be obtained and maintained by conventional methods. That is, by crossing male and female animals each having the DNA wherein the genetic nucleic acid sequence capable of expression for PP2Cε is knocked-out, homozygote animals having the inactivated DNA in both loci can be obtained. The homozygotes thus obtained may be reared so that one normal animal and two or more homozygotes are produced from a mother animal to efficiently obtain such homozygotes. By crossing male and female heterozygotes, homozygotes and heterozygotes having the inactivated DNA are proliferated and passaged.

Since the nonhuman mammal or cell strain, in which the genetic nucleic acid sequence capable of expression for PP2Cε in this embodiment is knocked-out, lacks the biological activity of AMPK, can thus be a model with AMPK-mediated signal-derived diseases, and is thus useful for investigating causes for and therapy for these diseases.

When the expression level having the genetic nucleic acid sequence capable of expression for PP2Cε is increased, there occur various diseases such as arteriosclerosis.

For example, when there is a patient showing an increase in the protein encoded by the genetic nucleic acid sequence capable of expression for PP2Cε, the protein in which the genetic nucleic acid sequence capable of expression for PP2Cε in this embodiment is knocked-out is administered to the patient to express the protein in this embodiment in the living body, whereby the dephosphorylation of AMPK in the patient can be regulated.

Where the protein in this embodiment is used as the prophylactic/therapeutic agents described above, the protein in this embodiment is administered directly to human or other warm-blooded animal; alternatively, the protein is inserted into an appropriate vector such as retrovirus vector, adenovirus vector, adenovirus-associated virus vector, etc. and then administered to human or other warm-blooded animal in a conventional manner. The protein in this embodiment may also be administered as an intact protein, or prepared into medicines together with physiologically acceptable carriers such as adjuvants to assist its uptake, which are administered by gene gun or through a catheter such as a hydrogel catheter.

### OTHER EMBODIMENTS

As described above, the present invention has been described by reference to the embodiment thereof, but a description constituting a part of this disclosure and the drawings should not be construed as limiting the invention. From this disclosure, various alternative embodiments, examples and used arts would be made apparent to those skilled in the art.

For example, modifications to the embodiment provide the following inventions: A protein or peptide wherein a genetic nucleic acid sequence capable of expression for PP2Cε is introduced. A nonhuman mammal or cell strain wherein a genetic nucleic acid sequence capable of expression for PP2Cε is introduced. A vector wherein a genetic nucleic acid sequence capable of expression for PP2Cε is introduced. A mouse wherein a genetic nucleic acid sequence capable of expression for PP2Cε is introduced. Use of protein phosphatase 2Cε (PP2Cε) having a dephosphorylase action on AMPK.

As described above, the present invention naturally encompasses various embodiments etc. not described herein. Accordingly, the technical scope of the invention shall be determined by only specific features in the claims, which are reasonable from the above description.

### EXAMPLES

### Example 1

### Creation of PP2Cε Knockout Animal

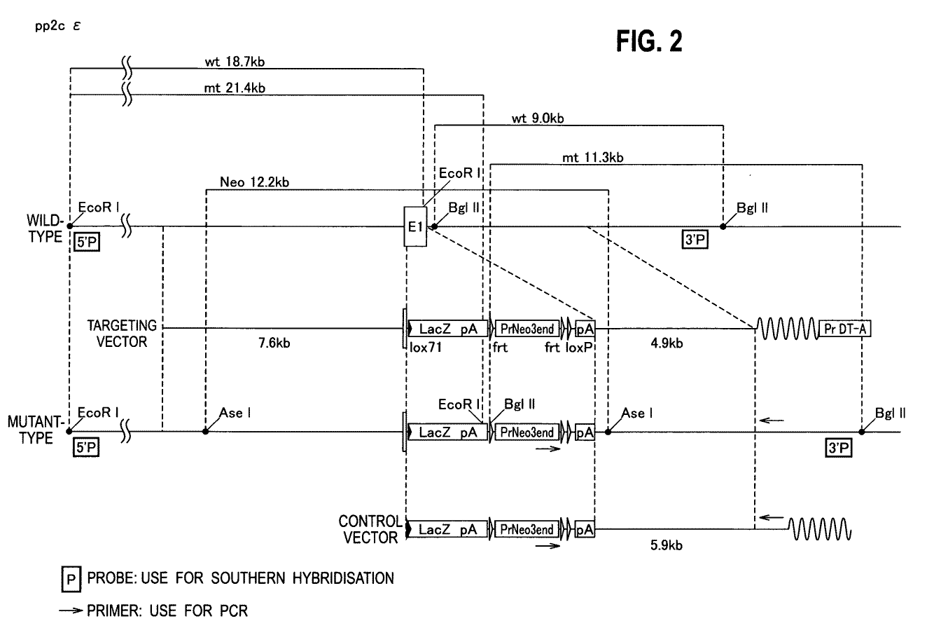

FIG. 2 shows construction and homologous recombination of a target (Targeting Vector/TV). As shown in FIG. 2, an EX1 region of PP2Cε gene was replaced by promoter-less lacZ gene (SEQ ID NO: 4) and a positive selection marker neomycin resistance gene (SEQ ID NO: 5), to crease a PP2Cε knockout mouse (SEQ ID NO: 1) wherein the PP2Cε locus was ruined.

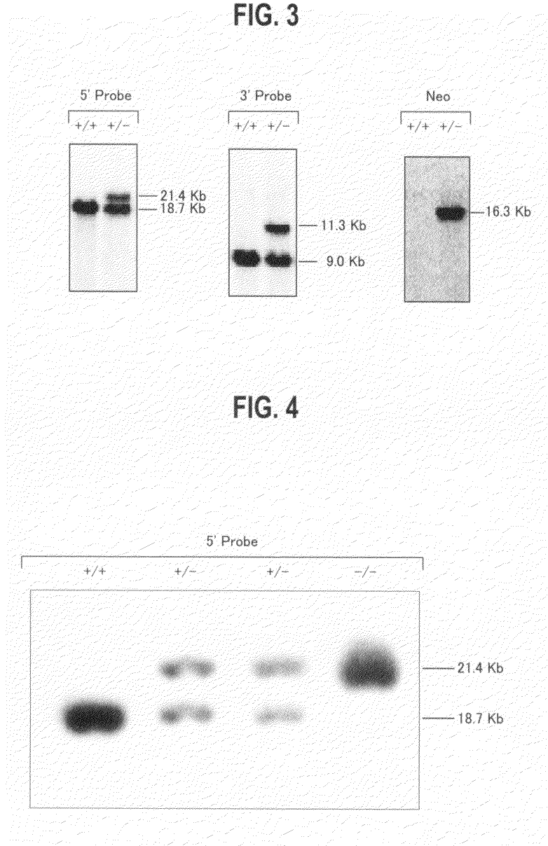

Southern blot analysis of homologously recombined ES clone was carried out under the following conditions. A 5′-probe was used for DNA digested with EcoRI, a 3′-probe was used for DNA digested with BgIII, and neomycin (Neo) probe was used for DNA digested with Ase. As ES cells, TT2 strain was used. FIG. 3 shows a profile in Southern blotting. DNAs in wild-type (+/+) and mutant ES cell (+/−) shown in FIG. 3 were confirmed to show signals at expected positions, respectively.

The DNA digested with EcoRI was analyzed in Southern blotting with the 5′-probe in genomic DNA purified from a mouse tail. FIG. 4 shows a profile in Southern blotting. DNAs in the wild-type (PP2C+/+), hetero (PP2C+/−) and homo (PP2C−/−) mice in FIG. 4 were confirmed to show signals at expected positions, respectively.

### Example 2

### Observation of Phenotype of PP2Cε Knockout Mouse

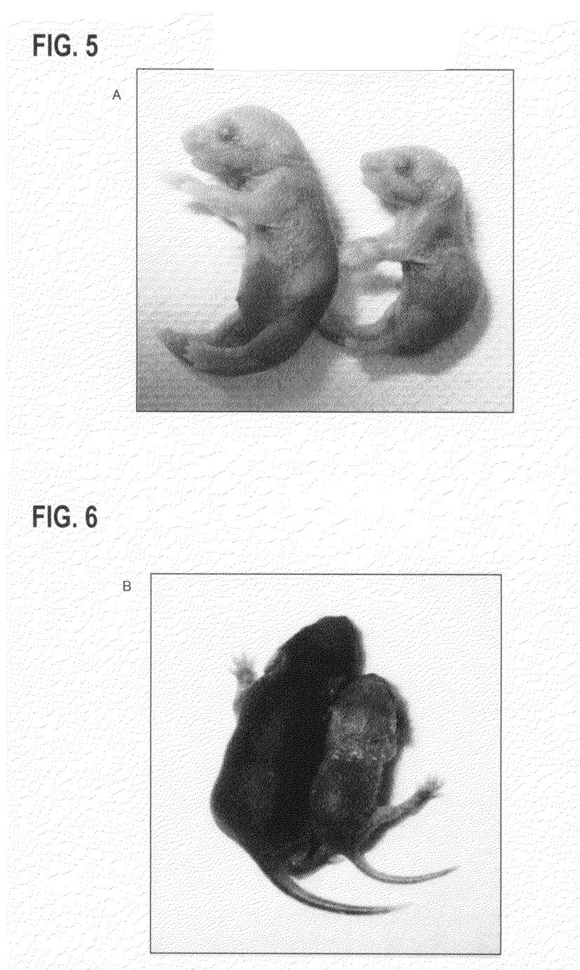

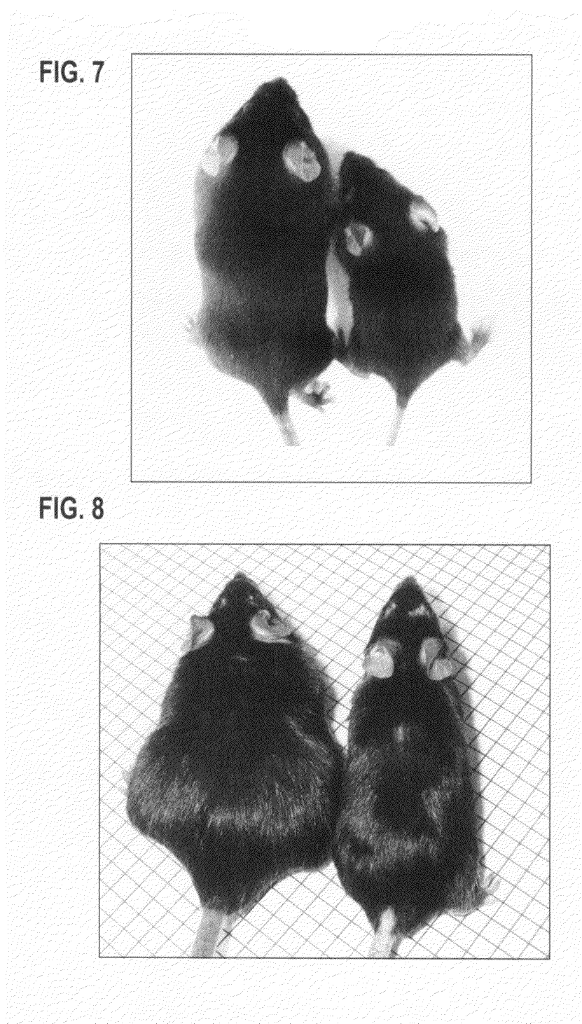

To confirm the phenotype of PP2Cε knockout mouse, the phenotype of wild-type (PP2Cε+/+) mouse was compared with that of homo (PP2C−/−) mouse. The photographs show wild-type (PP2Cε+/+) and homo (PP2Cε−/−) male litter mice within 24 hours after birth (FIG. 5) and one-week-old (FIG. 6), four-week-old (FIG. 7) and one-year-old (FIG. 8) wild-type (PP2Cε+/+) and homo (PP2Cε−/−) male litter mice, respectively. In FIGS. 5 to 8, the wild-type (PP2Cε+/+) male mouse shown in the left and the homo (PP2Cε−/−) male mouse in the right. As shown in FIGS. 5 to 8, the weight of the created homo (PP2Cε−/−) mice had been lighter from birth than the wild-type (PP2Cε+/+) litter mice.

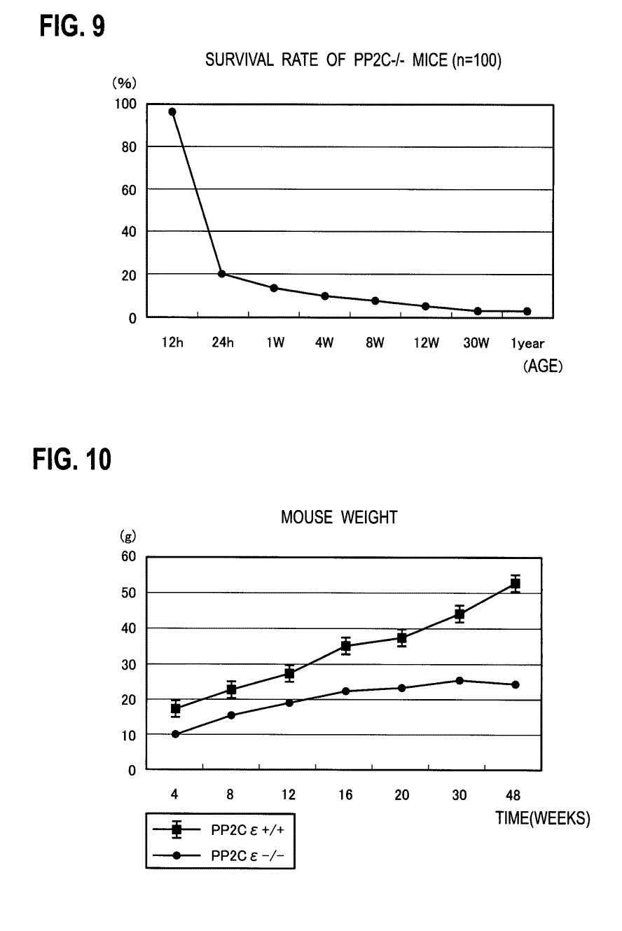

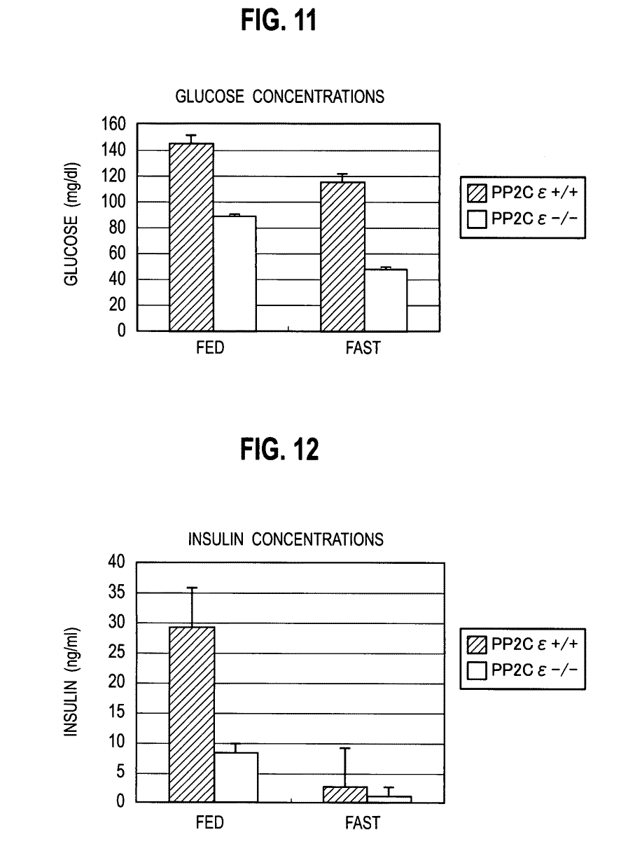

As shown in FIG. 9, the survival rate of the homo (PP2Cε−/−) mice were significantly lower, and the majority of the mice died within 24 hours. One-year-old PP2Cε−/− mice (3 cases) which could survive had normal reproductive capacities, but as shown in FIGS. 10, 11 and 12, their weight, blood sugar level and insulin level were lower than those of the wild-type.

### Example 3

### Influence of PP2Cε on Activation of AMPK (In Vitro Assay)

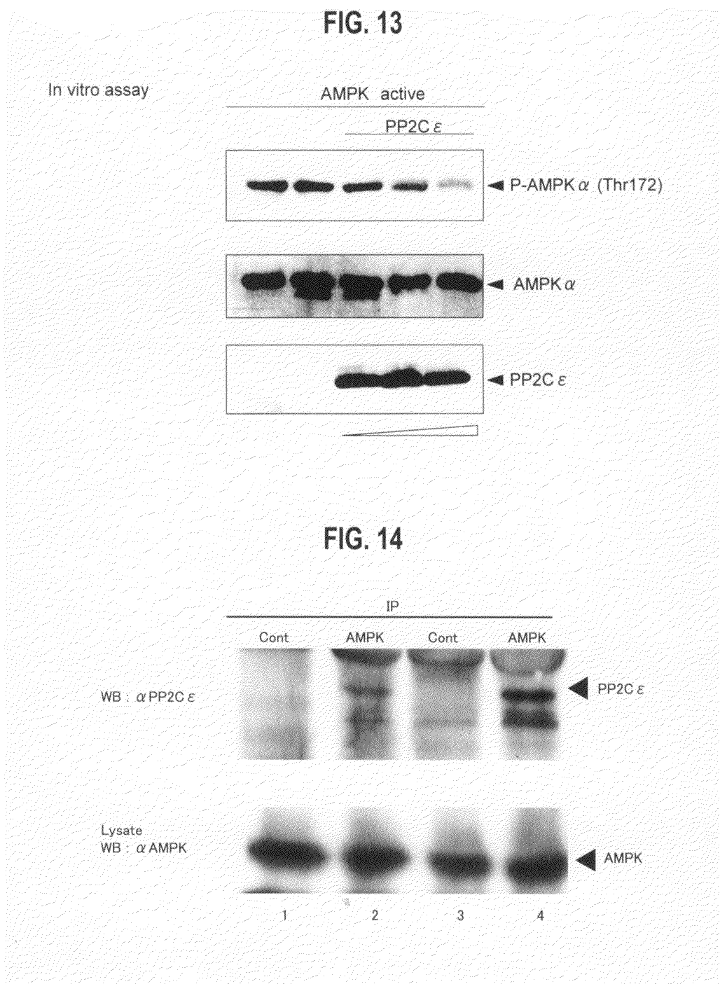

The influence of PP2Cε on activation of AMPK (in vitro assay) was examined. First, AMPK, active (upstate) protein, 10 μl (20 to 100 mU), and an ATP solution 10 μl were added to an AMPK reaction solution and shaken at 30° C. for 15 minutes. Thereafter, PP2Cε protein (0.8, 1.6, 2.4 μg) was added or not added to each sample and then shaken at 30° C. for 15 minutes. After SDS electrophoresis, Western blot analysis was conducted. The results are shown in FIG. 13. FIG. 13 shows that PP2Cε influences the activation of AMPK.

### Example 4

### Interaction Between PP2Cε and AMPK

FIG. 14 shows the interaction between endogenous PP2Cε and AMPK in the mouse liver.

From wild-type C57BL/6 male mice given a high-fat high-calorie food for 3 weeks, livers were obtained during eating or during 24-hour fasting, and extracts from the livers were immune-precipitated with anti-AMPKα antibody and detected with anti-PP2Cε antibody. The first and second lanes show the extracts of liver tissue excised during eating from the wild-type C57BL/6 male mice given a high-fat high calorie food. The third and fourth lanes show the extracts of liver tissue excised during fasting from the wild-type C57BL/6 male mice given a high-fat high calorie food.

The fourth upper lane in FIG. 14 shows that PP2Cε and AMPK are associated with each other. The second upper lane and the fourth upper lane in FIG. 14 show that PP2Cε and AMPK are associated more strongly during fasting than during eating.

### Example 5

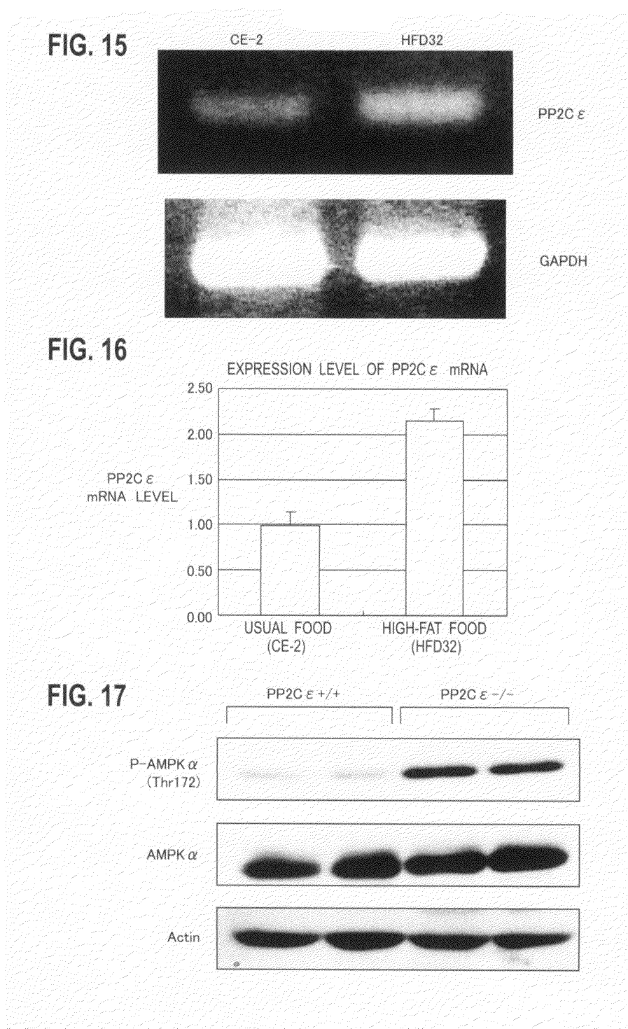

FIG. 15 shows a change in expression of mRNA for PP2Cε in mouse livers. The expression levels of mRNA for PP2Cε in the livers of wild-type C57BL/6 mice given a usual food (CE-2) or a high-fat food (HFD32) for 3 months were examined by RT-PCR.

FIG. 16 is a graph of expression level of mRNA for PP2Cε by mass spectrometric analysis. Eight-week-old wild-type C57BL/6 mice were given a usual food (CE-2) or a high-fat food (HFD32) for 3 weeks. From extracts of their livers, mRNA was extracted with RNeasy (QIAGEN) and subjected to reverse transcriptase reaction with a reverse transcriptase ReverTra Ace from TOYOBO and then subjected to RT-PCR with prepared PP2Cε and GAPDH primers. For mass spectrometric analysis in RT-PCR, Light Cycler manufactured by Roche was used.

From the above results, it was revealed that PP2Cε is expressed stronger when the mice were given the high-fat high calorie food (HFD32) than when the mice were given the usual food (CE-2).

### Example 6

For analysis at the organ level of the phenotype of homo (PP2Cε−/−) mice, the livers of wild-type (PP2Cε+/+) and homo (PP2C−/−) newborn litter mice were analyzed by Western blotting. The antibodies used were AMPK-α antibody, phosphor-AMPK-α (Thr172) antibody, AMPK-α antibody, phosphor-acetyl-CoA carboxylase (Ser79) antibody, acetyl CoA carboxylase antibody, phospho-mTOR (Ser2448) antibody, mTOR antibody, each of which was manufactured by Cell Signaling TECHNOLOGY, and actin (C-2) manufactured by Santa Cruz Biotechnology was used.

FIG. 17 shows the results of phosphorylation level of AMPKα Thr172 (upper stage), expression level of AMPKα protein (middle stage) and expression level of actin protein (lower stage) by Western blot analysis. The upper stage in FIG. 17 revealed that because of higher expression level of AMPKα protein, the activity of AMPK was higher in the homo (PP2Cε−/−) mice than in the wild-type (PP2Cε+/+) mice.

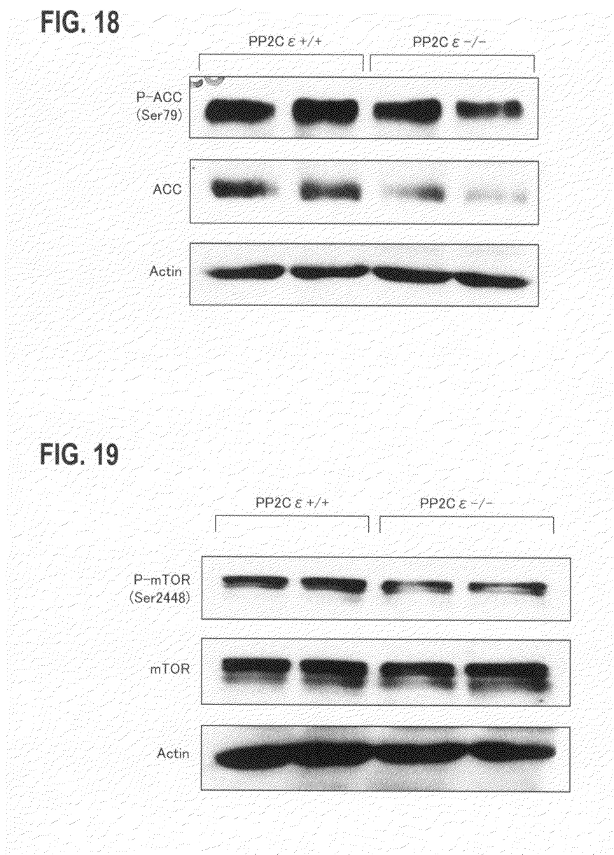

FIG. 18 shows the results of phosphorylation level of acetyl CoA carboxylase (ACC) Ser79 (upper stage), expression level of ACC protein (middle stage) and expression level of actin protein (lower stage) by Western blot analysis. The middle stage in FIG. 18 revealed that the expression level of ACC protein was higher in the homo (PP2Cε−/−) mice than in the wild-type (PP2Cε+/+) mice. It could be said that in the homo (PP2Cε−/−) mice, ACC was suppressed by activation of AMPK.

FIG. 19 shows the results of phosphorylation level of mammalian target of rapamycin (mTOR) Ser2448 (upper stage), expression level of mTOR protein (middle stage) and expression level of actin protein (lower stage) by Western blot analysis.

FIGS. 17 to 19 show acceleration of phosphorylation of AMPKα Thr172, down-regulation of acetyl CoA carboxylase (ACC) and acceleration of the phosphorylation thereof, and suppression of phosphorylation of mammalian target of rapamycin (mTOR).

From the Examples, it was revealed that when PP2Cε was made deficient, AMPK was activated by phosphorylation, which was followed by the inactivation of ACC1, ACC2 and mTOR downstream therefrom as well as by the activation of GLUT4 downstream therefrom, as shown in FIG. 1. Accordingly, it was estimated that the lower body weight of the PP2Cε−/− mice at birth and the lower survival rate thereof are attributable to a reduction in the activity of ACC1 and mTOR pathways, and the lower body weight of the 1-year-old mouse and lower blood sugar level and insulin level thereof are attributable to suppression of activation ACC2 and TORC2, acceleration of activation of GLUT4 and suppression of activation of mTOR. The up-regulation of PP2CE by ingestion of the high-fat high-calorie food suggested the presence of feedback regulatory mechanism on AMPK.

PP2Cε is essential for growth at the fetal stage in the mother's body, but with a high-fat high-calorie food given after birth, the expression of PP2Cε is increased thereby inactivating AMPK, which would lead to development of cancer and metabolic syndrome including obesity and disorder of sugar metabolism. In the future, the suppression of expression of PP2Cε by adenovirus or by SNALP developed in the last year can be expected to enhance the activation of AMPK and to induce suppression of obesity and ameliorate diabetes, and can be expected to contribute significantly to creation of an AMPK activation molecular target drug.

PP2Cε is a phosphatase for AMPK and has an inactivation action on AMPK. Suppression of expression of PP2Cε or suppression of binding of PP2Cε to AMPK promotes phosphorylation of AMPK thereby activating AMPK more significantly than by AICAR, metformin and a thiazolidine derivative (TZD), and is considered to contribute to weight loss, treatment of type 2 diabetes, suppression of metabolic syndrome, and suppression of generation of cancer (see Trends Pharmacol Sci., Vol. 26 (2005), pp. 69-76).

With a high-fat high-calorie food given, the homo (PP2Cε−/−) mice did not show an increase in body weight and blood sugar level as compared with those of the wild-type (PP2Cε+/+) mice. The 1-year-old homo (PP2Cε−/−) mice showed a lower body weight and blood sugar level and a significantly lower insulin level than those of the wild-type (PP2Cε+/+) mice. From the foregoing, PP2Cε when deprived of its action as a phosphatase for AMPK can be sufficiently expected for use not only as a therapeutic agent for diabetes but also as a prophylactic agent for metabolic syndrome and cancer.

The SEQUENCE LISTING in this specification shows the following sequences:

SEQ ID NO: 1 shows a nucleotide sequence of PP2Cε knockout mouse in Example 1.

SEQ ID NO: 2 shows a mouse primer used in Example 1.

SEQ ID NO: 3 shows a mouse primer used in Example 1.

SEQ ID NO: 4 shows lacZ gene used in Example 1.

SEQ ID NO: 5 shows a neomycin resistance gene used in Example 1.

In the specification, the codes of bases are denoted in accordance with the IUPAC-IUB Commission on Biochemical Nomenclature or by the common codes in the art, examples of which are shown below.

A: adenine
T: thymine
G: guanine
C: cytosine

[Sequence List]

2007041617290057865264 4623346e 061208002J000021.app.txt

What is claimed is:

1. A drug comprising:
RNA interference with protein phosphatase 2Cε (PP2Cε) as an active ingredient.

2. The drug according to claim 1, wherein the RNA interference with PP2Cε is selected from the group consisting of RNAi, siRNA, and shRNA.

3. The drug according to claim 1, wherein the drug is used for prophylaxis and therapy of AMPK-mediated signal-derived diseases.

4. The drug according to claim 2, wherein the drug is used for prophylaxis and therapy of AMPK-mediated signal-derived diseases.

5. The drug according to claim 1, wherein the drug is used in regulation of dephosphorylation of AMPK.

6. The drug according to claim 2, wherein the drug is used in regulation of dephosphorylation of AMPK.

7. A drug comprising:
a vector, wherein a genetic nucleic acid sequence capable of expression for PP2Cε is knocked-out.

8. The drug according to claim 7, wherein the drug is used for prophylaxis and therapy of AMPK-mediated signal-derived diseases.

9. The drug according to claim 7, wherein the drug is used in regulation of dephosphorylation of AMPK.

10. A therapeutic method for treating AMPK-mediated signal-derived diseases in nonhuman mammals, comprising:
inhibiting the association of protein phosphatase 2Cε (PP2Cε) with AMP kinase (AMPK).

11. A protein, comprising a genetic nucleic acid sequence, wherein a genetic nucleic acid sequence capable of expression for PP2Cε is knocked-out.

12. A peptide, comprising a genetic nucleic acid sequence, wherein a genetic nucleic acid sequence capable of expression for PP2Cε is knocked-out.

13. A nonhuman mammal, comprising a genetic nucleic acid sequence, wherein a genetic nucleic acid sequence capable of expression for PP2Cε is knocked-out.

14. A cell strain, comprising a genetic nucleic acid sequence, wherein a genetic nucleic acid sequence capable of expression for PP2Cε is knocked-out.

15. A vector, comprising a genetic nucleic acid sequence, wherein a genetic nucleic acid sequence capable of expression for PP2Cε is knocked-out.

16. A mouse, comprising a genetic nucleic acid sequence, wherein a genetic nucleic acid sequence capable of expression for PP2Cε is knocked-out.

17. A use of protein phosphatase 2Cε (PP2Cε) as a phosphatase that directly dephosphorylates and activates AMPK.
